SEQUENCE LISTING (40 sequences)

SEQ ID NO: 1 - RNA, 77 nt, synthetic construct
  source 1..77: mol_type = other RNA
ccggcggtagttcagcagggcagaacggcggactctaaatccgcatggcgccggttcaaatccggcccgccggacca

SEQ ID NO: 2 - RNA, 76 nt, synthetic construct
  source 1..76: mol_type = other RNA
ccggcggtagttcagagggcagaacggcggactctaaatccgcatggcgccagttcaaatctggcccgccggacca

SEQ ID NO: 3 - RNA, 76 nt, synthetic construct
  source 1..76: mol_type = other RNA
cccgccttagttcagagggcagaacggcggactctaaatccgcatggcgccggttcaaatccggcaggcgggacca

SEQ ID NO: 4 - RNA, 76 nt, synthetic construct
  source 1..76: mol_type = other RNA
ccggcggtagttcagagggcagaacggcggactctaaatccgcatggcgccggttcaaatccggcccgccggacca

SEQ ID NO: 5 - RNA, 76 nt, synthetic construct
  source 1..76: mol_type = other RNA
cccgccttagttcagagggcagaacggcggactctaaatccgcatggcacgggttcaaatcccgtaggcgggacca

SEQ ID NO: 6 - RNA, 76 nt, synthetic construct
  source 1..76: mol_type = other RNA
ccggcggtagttcagagggcagaacggcggactctaaatccgcatggcaggggttcaaatcccctccgccggacca

SEQ ID NO: 7 - RNA, 77 nt, synthetic construct
  source 1..77: mol_type = other RNA
cccgccttagttcagcagggcagaacggcggactctaaatccgcaggtcgccggttcaaatccggcaggcgggacca

SEQ ID NO: 8 - RNA, 76 nt, synthetic construct
  source 1..76: mol_type = other RNA
cccgccttagttcagagggcagaacggcggactctaaatccgcaggtcgccggttcaaatccggcaggcgggacca

SEQ ID NO: 9 - RNA, 77 nt, synthetic construct
  source 1..77: mol_type = other RNA
cccgccttagttcagcagggcagaacggcggactctaaatccgcaggtcacgggttcaaatcccgtaggcgggacca

SEQ ID NO: 10 - RNA, 76 nt, synthetic construct
  source 1..76: mol_type = other RNA
ccggcggtagttcagagggcagaacggcggactctaaatccgcatggcacgggttcaaatcccgtccgccggacca

SEQ ID NO: 11 - RNA, 76 nt, synthetic construct
  source 1..76: mol_type = other RNA
ccggcggtagttcagagggcagaacggcggactctaaatccgcaggtcaggggttcaaatcccctccgccggacca

SEQ ID NO: 12 - RNA, 77 nt, synthetic construct
  source 1..77: mol_type = other RNA
ccggcggtagttcagcagggcagaacggcggactctaaatccgcatggcaggggttcaaatcccctccgccggacca

SEQ ID NO: 13 - RNA, 76 nt, synthetic construct
  source 1..76: mol_type = other RNA
ccggcggtagttcagagggcagaacggcggactctaaatccgcaggtcacgggttcaaatcccgtccgccggacca

SEQ ID NO: 14 - RNA, 77 nt, synthetic construct
  source 1..77: mol_type = other RNA
ccggaggtagttcagcagggcagaacggcggactctaaatccgcatggcgccggttcaaatccggcccttcggacca

SEQ ID NO: 15 - RNA, 76 nt, synthetic construct
  source 1..76: mol_type = other RNA
ccggtgttagttcagagggcagaacggcggactctaaatccgcatgtcacgggttcaaatcccgtacaccggacca

SEQ ID NO: 16 - RNA, 76 nt, synthetic construct
  source 1..76: mol_type = other RNA
ccgaccttagttcagagggcagaacggcggactctaaatccgcatgtcaggggttcaaatcccctaggtcggacca

SEQ ID NO: 17 - DNA, 76 bp, synthetic construct
  source 1..76: mol_type = other DNA
ccggcggtagttcagagggcagaacggcggactctaaatccgcatggcgccagttcaaatctggcccgccggacca

SEQ ID NO: 18 - DNA, 76 bp, synthetic construct
  source 1..76: mol_type = other DNA
cccgccttagttcagagggcagaacggcggactctaaatccgcatggcgccggttcaaatccggcaggcgggacca

SEQ ID NO: 19 - DNA, 76 bp, synthetic construct
  source 1..76: mol_type = other DNA
ccggcggtagttcagagggcagaacggcggactctaaatccgcatggcgccggttcaaatccggcccgccggacca

SEQ ID NO: 20 - DNA, 76 bp, synthetic construct
  source 1..76: mol_type = other DNA
cccgccttagttcagagggcagaacggcggactctaaatccgcatggcacgggttcaaatcccgtaggcgggacca

SEQ ID NO: 21 - DNA, 76 bp, synthetic construct
  source 1..76: mol_type = other DNA
ccggcggtagttcagagggcagaacggcggactctaaatccgcatggcaggggttcaaatcccctccgccggacca

SEQ ID NO: 22 - DNA, 77 bp, synthetic construct
  source 1..77: mol_type = other DNA
cccgccttagttcagcagggcagaacggcggactctaaatccgcaggtcgccggttcaaatccggcaggcgggacca

SEQ ID NO: 23 - DNA, 76 bp, synthetic construct
  source 1..76: mol_type = other DNA
cccgccttagttcagagggcagaacggcggactctaaatccgcaggtcgccggttcaaatccggcaggcgggacca

SEQ ID NO: 24 - DNA, 77 bp, synthetic construct
  source 1..77: mol_type = other DNA
cccgccttagttcagcagggcagaacggcggactctaaatccgcaggtcacgggttcaaatcccgtaggcgggacca

SEQ ID NO: 25 - DNA, 76 bp, synthetic construct
  source 1..76: mol_type = other DNA
ccggcggtagttcagagggcagaacggcggactctaaatccgcatggcacgggttcaaatcccgtccgccggacca

SEQ ID NO: 26 - DNA, 76 bp, synthetic construct
  source 1..76: mol_type = other DNA
ccggcggtagttcagagggcagaacggcggactctaaatccgcaggtcaggggttcaaatcccctccgccggacca

SEQ ID NO: 27 - DNA, 77 bp, synthetic construct
  source 1..77: mol_type = other DNA
ccggcggtagttcagcagggcagaacggcggactctaaatccgcatggcaggggttcaaatcccctccgccggacca

SEQ ID NO: 28 - DNA, 76 bp, synthetic construct
  source 1..76: mol_type = other DNA
ccggcggtagttcagagggcagaacggcggactctaaatccgcaggtcacgggttcaaatcccgtccgccggacca

SEQ ID NO: 29 - DNA, 77 bp, synthetic construct
  source 1..77: mol_type = other DNA
ccggaggtagttcagcagggcagaacggcggactctaaatccgcatggcgccggttcaaatccggcccttcggacca

SEQ ID NO: 30 - DNA, 76 bp, synthetic construct
  source 1..76: mol_type = other DNA
ccggtgttagttcagagggcagaacggcggactctaaatccgcatgtcacgggttcaaatcccgtacaccggacca

SEQ ID NO: 31 - DNA, 76 bp, synthetic construct
  source 1..76: mol_type = other DNA
ccgaccttagttcagagggcagaacggcggactctaaatccgcatgtcaggggttcaaatcccctaggtcggacca

SEQ ID NO: 32 - RNA, 77 nt, synthetic construct
  source 1..77: mol_type = other RNA
ccggcggtagttcagcctggtagaacggcggactgtagatccgcatgtcgctggttcaaatccggcccgccggacca

SEQ ID NO: 33 - DNA, 77 bp, synthetic construct
  source 1..77: mol_type = other DNA
ccggcggtagttcagcctggtagaacggcggactgtagatccgcatgtcgctggttcaaatccggcccgccggacca

SEQ ID NO: 34 - DNA, 77 bp, synthetic construct
  source 1..77: mol_type = other DNA
cnngcnntagttcagcagggcagaacggcggactctaaatccgcatggcwnrggttcaaatccynwnngcnngacca

SEQ ID NO: 35 - protein, 306 aa, synthetic construct
MDEFEMIKRNTSEIISEEELREVLKKDEKSAVIGFEPSGKIHLGHYLQIKKMIDLQNAGFDIIIYLADLHAYLNQKGELDEIRKIGDYNKKVFEAMGLKAKYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 36 - RNA, 76 nt, synthetic construct
  source 1..76: mol_type = other RNA
  variation 16: note = May be absent
ccsrhsktagttcagcagggcagaacggcggactctaaatccgcakgkcrsnrgtcaaatcynsymsdysggacca

SEQ ID NO: 37 - RNA, 77 nt, synthetic construct
  source 1..77: mol_type = other RNA
ccggcggtagttcagcagggcagaacggcggactctaaatccgcatggcgctggttcaaatccagcccgccggacca

SEQ ID NO: 38 - RNA, 77 nt, synthetic construct
  source 1..77: mol_type = other RNA
ccggcggtagttcagcagggcagaacggcggactctaaatccgcatggcgcaggttcaaatcctgcccgccggacca

SEQ ID NO: 39 - protein, 306 aa, synthetic construct
MDEFEMIKRNTSEIVSEEELREVLKKDEKSAVIGFEPSGKIHLGHYLQIKKMIDLQNAGFDIIIYLADLHAYLNQKGELDEIRKIGDYNKKVFEAMGLKAKYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 40 - RNA, 10 nt, synthetic construct
  source 1..10: mol_type = other RNA
cagagggcag

(19) United States
(12) Patent Application Publication
(10) Pub. No.: US 2024/0263209 A1
(43) Pub. Date: Aug. 8, 2024

(54) COMPOSITION OF TRANSFER RNAS AND USE IN PRODUCTION OF PROTEINS CONTAINING NON-STANDARD AMINO ACIDS

(71) Applicant: Absci Corporation, Vancouver, WA (US)

(72) Inventors: Yi Zhang, Vancouver, WA (US); Matthew Weinstock, Vancouver, WA (US); Miles Gander, Vancouver, WA (US)

(21) Appl. No.: 18/606,513

(22) Filed: Mar. 15, 2024

Related U.S. Application Data
(63) Continuation of application No. PCT/US2022/076575, filed on Sep. 16, 2022.
(60) Provisional application No. 63/245,789, filed on Sep. 17, 2021.

(51) Int. Cl. C12P 21/00; C12N 9/00; C12N 15/11; C12N 15/70; C12R 1/19

(52) U.S. Cl. CPC C12P 21/00; C12N 9/93; C12N 15/11; C12N 15/70; C12R 2001/19; C12Y 601/01001

(57) ABSTRACT

This disclosure provides engineered tRNAs and the corresponding aminoacyl tRNA synthetase for efficient production of proteins containing non-standard amino acids. These engineered orthogonal tRNA (O-tRNA)/orthogonal aminoacyl tRNA synthetase (O-RS) pair, i.e., Orthogonal Translation Systems (OTSs) can be used to incorporate a non-standard amino acid in a specific position in a growing polypeptide in response to a selector codon that is recognized by the engineered tRNA.

### CROSS REFERENCE TO RELATED APPLICATIONS

This application is a continuation of PCT/US2022/076575, filed on Sep. 16, 2022, which claims the benefit of and priority to U.S. Provisional Application No. 63/245,789, filed Sep. 17, 2021, which is hereby incorporated by reference in its entirety.

### SEQUENCE LISTING

The instant application contains a Sequence Listing which is hereby incorporated by reference in its entirety. Said XML copy, created on Sep. 15, 2022, is named ABS-018WO_SL.xml, and is 39,224 bytes in size.

### BACKGROUND

Although proteins are ubiquitously involved in almost all biological processes, the vast majority of them are biosynthesized from only 20 standard amino acids with a limited set of functional groups (amines, carboxylic acids, amides, alcohols, thiols, etc.). One goal of synthetic biology is to expand the chemical alphabets utilized by living organisms. The ability to incorporate non-standard amino acids (nsAAs) into proteins is a highly desirable feature for a protein expression system as it enables novel chemistry which streamlines the production of previously difficult-to-produce molecules. For example, incorporation of nsAAs into biologics that support site-specific conjugation under mild aqueous conditions would provide a highly simplified route for the attachment of half-life extension moieties (e.g., PEG), uniform glycan structures, and antibody-drug conjugates (ADCs).

The incorporation of nsAAs into proteins and peptides is typically achieved by the expression of an engineered transfer RNA (tRNA)/aminoacyl tRNA synthetase pair, i.e., Orthogonal Translation Systems (OTSs), that can aminoacylate the nsAA of interest onto the novel tRNA. To achieve efficient production of the desired protein containing nsAA, the engineered tRNA must be selectively aminoacylated by its cognate aminoacyl-tRNA synthetase (aaRS) while remaining inactive to all endogenous aaRSs in the protein expression system. The resulting aminoacyl-tRNA must be efficiently recognized by elongation factor Tu (EF-Tu) to be translocated to the A site of ribosome; after binding to the ribosomal A site, the aminoacyl-tRNA must function efficiently in translation as a substrate for peptidyl transferase; and finally the tRNA bearing the growing peptide chain must be translocated to the P site, undergo another acyl transfer reaction, and be released from the ribosome.

The performance of an OTS can be defined in terms of two main parameters, fidelity and efficiency. Fidelity refers to the accuracy with which the reassigned codon is read through by the ribosome as the nsAA versus misread as any other amino acid. A lower fidelity OTS will result in a mixture of proteins harboring nsAA and one or more other amino acids at each reassigned codon, which significantly complicates the purification process or even renders the separation impossible. Efficiency reflects how effectively the ribosome is able to read through the reassigned codon in the presence of the OTS and the nsAA. A less efficient nsAA-incorporation system will result in a lower yield of the protein of interest. A desirable OTS should have a balanced profile in terms of both fidelity and efficiency during the entire course of fermentation. Ideally, the mischarging of standard amino acid is negligible while the read through efficiency is close to if not better than translation of the wild type DNA sequence.

OTSs of various sources have been engineered and used for production of proteins containing nsAA in bacterial protein expression systems. For example, OTSs from archaea Methanococcus jannaschii were engineered for a number of new amino acids with novel chemical, physical or biological properties, including photoaffinity labels and photoisomerizable amino acids, photocrosslinking amino acids (see Chin, J. W., et al. (2002) Proc. Natl. Acad. Sci. U.S.A. 99:11020-11024; and, Chin, J. W., et al., (2002) J. Am. Chem. Soc. 124:9026-9027), keto amino acids (see, Wang, L., et al., (2003) Proc. Natl. Acad. Sci. U.S.A. 100:56-61 and Zhang, Z. et al., Biochem. 42(22):6735-6746 (2003)), heavy atom containing amino acids, and glycosylated amino acids have been incorporated efficiently and with high fidelity into proteins in Escherichia coli in response to the amber codon (TAG). Several other orthogonal pairs have been reported, including: glutaminyl systems (see, e.g., Liu, D. R., and Schultz, P. G. (1999) Proc. Natl. Acad. Sci. U.S.A. 96:4780-4785), aspartyl systems (see, e.g., Pastrnak, M., et al., (2000) Helv. Chim. Acta 83:2277 2286), tyrosyl systems (see, e.g., Ohno, S., et al., (1998). J. Bio Chem. (Tokyo, Jpn.) 124:1065-1068; and Kowal, A. K., et al., (2001) Proc. Natl. Acad. Sci. U.S.A. 98:2268-2273), and systems derived from S. cerevisiae tRNAs and synthetases have been described for the potential incorporation of non-standard amino acids in E. coli.

Although non-standard amino acids are typically incorporated into proteins with acceptable efficiency and fidelity, further systemic optimization for improved protein yields is highly desirable. The extent of incorporation of the non-standard amino acid varies-only in rare cases can be quantitative, which results in suboptimal quantity and quality profile of the product. Prior known OTSs showcased either reduced fidelity or efficiency during high cell-density fermentation of bacterial expression system. These defects are particularly pronounced in highly optimized bacterial expression systems which are dedicated to produce biologics. Therefore, to further expand the application scope of the nsAA, there is a need to develop improved and/or additional components of the OTSs, e.g., tRNAs.

### SUMMARY

In certain aspects, disclosed herein is an orthogonal tRNA (O-tRNA) comprising a nucleic acid sequence at least 85% identical to the sequence set forth in SEQ ID NO: 1 and comprising a deletion of the cytosine located at nucleic acid position 16 of the O-tRNA, wherein the nucleic acid positions correspond to the sequence set forth in SEQ ID NO: 1; and wherein the O-tRNA is capable of being aminoacylated with at least one non-standard amino acid (nsAA) by an orthogonal aminoacyl tRNA synthetase (O-RS). In certain embodiments, the nucleic acid sequence is at least 90% identical, at least 91% identical, at least 92% identical, at least 93% identical, at least 94% identical, at least 95% identical, at least 96% identical, at least 97% identical, at least 98% identical, or at least 98.7% identical to the sequence set forth in SEQ ID NO: 1. In certain embodiments, the O-tRNA comprises an adenine at nucleic acid position 53 and a uracil at nucleic acid position 63, wherein the nucleic acid positions correspond to the sequence set forth in SEQ ID NO: 1. In certain embodiments, the O-tRNA comprises a nucleic acid sequence consisting of the sequence set forth in SEQ ID NO: 2. In certain embodiments, the O-tRNA comprises a cytosine at nucleic acid positions 3 and 6; a uracil at nucleic acid position 7, an adenosine at nucleic acid position 67, and a guanine at nucleic acid positions 68 and 71, wherein the nucleic acid positions correspond to the sequence set forth in SEQ ID NO: 1. In certain embodiments, the O-tRNA comprises a nucleic acid sequence consisting of the sequence set forth in SEQ ID NO: 3. In certain embodiments, the O-tRNA comprises a nucleic acid sequence consisting of the sequence set forth in SEQ ID NO: 4. In certain embodiments, the O-tRNA comprises the sequence CAG-AGGGCAG at nucleic acid positions 13 to 23, wherein the nucleic acid positions correspond to the sequence set forth in SEQ ID NO: 1.

In certain embodiments, the O-tRNA comprises a nucleic acid sequence consisting of a sequence set forth in SEQ ID NO: 36, wherein the sequence does not comprise SEQ ID NO: 1, SEQ ID NO: 37, or SEQ ID NO: 38. In certain embodiments, the O-tRNA comprises a cytosine at position 3. In certain embodiments, the O-tRNA comprises an adenine at position 4. In certain embodiments, the O-tRNA comprises a uracil at position 5. In certain embodiments, the O-tRNA comprises a cytosine at position 6. In certain embodiments, the O-tRNA comprises a uracil at position 7. In certain embodiments, the O-tRNA comprises a guanine at position 46. In certain embodiments, the O-tRNA comprises a uracil at position 48. In certain embodiments, the O-tRNA comprises an adenine at position 50. In certain embodiments, the O-tRNA comprises a guanine at position 51. In certain embodiments, the O-tRNA comprises an adenine at position 53. In certain embodiments, the O-tRNA comprises a uracil at position 63. In certain embodiments, the O-tRNA comprises a cytosine at position 64. In certain embodiments, the O-tRNA comprises a cytosine at position 65. In certain embodiments, the O-tRNA comprises a uracil at position 66. In certain embodiments, the O-tRNA comprises an adenine at position 67. In certain embodiments, the O-tRNA comprises a guanine at position 68. In certain embodiments, the O-tRNA comprises an adenine or a uracil at position 69. In certain embodiments, the O-tRNA comprises a uracil at position 70. In certain embodiments, the O-tRNA comprises a guanine at position 71. In certain embodiments, the O-tRNA comprises a cytosine at position 3, a cytosine at position 6, and a uracil at position 7. In certain embodiments, the O-tRNA comprises a guanine at position 46 and a uracil at position 48. In certain embodiments, the O-tRNA comprises an adenine at position 67, a guanine at position 68, and a guanine at position 71.

In certain embodiments, the O-tRNA comprises a nucleic acid sequence consisting of a sequence set forth in SEQ ID NOs: 2-16. In certain embodiments, the O-tRNA is aminoacylated. In certain embodiments, the O-tRNA is aminoacylated with an nsAA. In certain embodiments, the nsAA has the structure according to Formula I; wherein the R group is any substituent other than a corresponding substituent used in the twenty natural amino acids. In certain embodiments, the nsAA has the structure according to Formula I wherein the R group comprises an alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amine, or combinations thereof. In certain embodiments, the nsAA is selected from the group consisting of: an amino acid comprising a photoactivatable cross-linker, a spin-labeled amino acid, a fluorescent amino acid, a metal binding amino acid, a metal containing amino acid, a radioactive amino acid, an amino acid comprising at least one novel functional group, an amino acid that covalently or noncovalently interacts with other molecules, a photocaged amino acid, a photoisomerizable amino acid, an amino acids comprising biotin or a biotin analogue, a carbohydrate-modified amino acid, an amino acid comprising polyethylene glycol or polyether, a heavy atom substituted amino acid, a chemically cleavable amino acid, a photocleavable amino acid, and combinations thereof. In certain embodiments, the nsAA comprises a tyrosine analog. In certain embodiments, the tyrosine analog is selected from the group consisting of a para-substituted tyrosine, an ortho-substituted tyrosine, and a meta-substituted tyrosine. In certain embodiments, the substituted tyrosine comprises a keto group, an acetyl group, a benzoyl group, an amino group, a hydrazine, a hydroxyamine, a thiol group, a carboxy group, an isopropyl group, a methyl group, a branched hydrocarbon, a saturated or unsaturated hydrocarbon, an O-methyl group, a polyether group, a nitro group, or combinations thereof. In certain embodiments, the nsAA comprises a glutamine analog. In certain embodiments, the glutamine analog comprises an α-hydroxy derivative, a T-substituted derivative, a cyclic derivative, or an amide substituted glutamine derivative. In certain embodiments, the nsAA comprises a phenylalanine analog. In certain embodiments, the phenylalanine analog is an amino-, an isopropyl-, or an O-allyl-containing phenylalanine analog. In certain embodiments, the phenylalanine analog is selected from the group consisting of a para-substituted phenylalanine, an ortho-substituted phenylalanine, and a meta-substituted phenylalanine. In certain embodiments, the substituent comprises a hydroxy group, a methoxy group, a methyl group, an allyl group, an aldehyde, an azido, an iodo, a bromo, a keto group or an acetyl group. In certain embodiments, the nsAA comprises a para-acetyl phenylalanine.

In certain embodiments, the nsAA is selected from the group consisting of a p-propargyl phenylalanine, an O-methyl-L-tyrosine, an L-3-(2-naphthyl)alanine, a 3-methylphenylalanine, an O-4-allyl-L-tyrosine, a 4-propyl-L-tyrosine, a tri-O-acetyl-GlcNAcB-serine, an L-Dopa, a fluorinated phenylalanine, an isopropyl-L-phenylalanine, a p-acyl-L-phenylalanine, a p-benzoyl-L-phenylalanine, an L-phosphoserine, a phosphono serine, a phosphonotyrosine, a p-iodo-phenylalanine, a p-bromophenylalanine, a p-amino-L-phenylalanine, and an isopropyl-L-phenylalanine. In certain embodiments, the nsAA is selected from the group consisting of 4-acetyl-phenylalanine (AcF), 4-azido-phenylalanine (AzF); 4-propargyloxyphenylalanine (PaF); and 4-aminophenylalanine (AmF). In certain embodiments, the nsAA comprises an 4-acetyl-phenylalanine (AcF). In certain embodiments, the nsAA comprises an 4-azido-phenylalanine (AzF). In certain embodiments, the nsAA comprises an 4-propargyloxyphenylalanine (PaF). In certain embodiments, the nsAA comprises an 4-aminophenylalanine (AmF).

In certain embodiments, the O-tRNA is chemically aminoacylated. In certain embodiments, the O-tRNA is enzymatically aminoacylated. In certain embodiments, the O-tRNA is enzymatically aminoacylated by a ribozyme. In certain embodiments, the O-tRNA is derived from archaeal tRNA. In certain embodiments, the O-tRNA is derived from M. jannaschii.

In certain aspects, disclosed herein is an orthogonal tRNA synthetase (O-RS) comprising the amino acid sequence consisting of the sequence set forth in SEQ ID NO: 39. In certain aspects, disclosed herein is an orthogonal translation system (OTS) comprising the O-tRNA and an O-RS disclosed herein.

In certain embodiments, the O-RS comprises an O-RS of an M. jannaschii tyrosyl-tRNA synthetase. In certain embodiments, the O-RS comprises an amino acid sequence consisting of the sequence set forth in SEQ ID NO: 35 or 39. In certain embodiments, the OTS further comprises the nsAA. In certain embodiments, the nsAA has the structure according to Formula I; wherein the R group is any substituent other than a corresponding substituent used in the twenty natural amino acids. In certain embodiments, the nsAA has the structure according to Formula I wherein the R group comprises an alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amine, or combinations thereof. In certain embodiments, the nsAA is selected from the group consisting of: an amino acid comprising a photoactivatable cross-linker, a spin-labeled amino acid, a fluorescent amino acid, a metal binding amino acid, a metal containing amino acid, a radioactive amino acid, an amino acid comprising at least one novel functional group, an amino acid that covalently or noncovalently interacts with other molecules, a photocaged amino acid, a photoisomerizable amino acid, an amino acids comprising biotin or a biotin analogue, a carbohydrate-modified amino acid, an amino acid comprising polyethylene glycol or polyether, a heavy atom substituted amino acid, a chemically cleavable amino acid, a photocleavable amino acid, and combinations thereof. In certain embodiments, the nsAA comprises a tyrosine analog. In certain embodiments, the tyrosine analog is selected from the group consisting of a para-substituted tyrosine, an ortho-substituted tyrosine, and a meta-substituted tyrosine. In certain embodiments, the substituted tyrosine comprises a keto group, an acetyl group, a benzoyl group, an amino group, a hydrazine, a hydroxyamine, a thiol group, a carboxy group, an isopropyl group, a methyl group, a branched hydrocarbon, a saturated or unsaturated hydrocarbon, an O-methyl group, a polyether group, a nitro group, or combinations thereof. In certain embodiments, the nsAA comprises a glutamine analog. In certain embodiments, the glutamine analog comprises an α-hydroxy derivative, a T-substituted derivative, a cyclic derivative, or an amide substituted glutamine derivative. In certain embodiments, the nsAA comprises a phenylalanine analog. In certain embodiments, the phenylalanine analog is an amino-, an isopropyl-, or an O-allyl-containing phenylalanine analog. In certain embodiments, the phenylalanine analog is selected from the group consisting of a para-substituted phenylalanine, an ortho-substituted phenylalanine, and a meta-substituted phenylalanine. In certain embodiments, the substituent comprises a hydroxy group, a methoxy group, a methyl group, an allyl group, an aldehyde, an azido, an iodo, a bromo, a keto group or an acetyl group. In certain embodiments, the nsAA comprises a para-acetyl phenylalanine.

In certain embodiments, the nsAA of the OTS is selected from the group consisting of a p-propargyl phenylalanine, an O-methyl-L-tyrosine, an L-3-(2-naphthyl)alanine, a 3-methylphenylalanine, an O-4-allyl-L-tyrosine, a 4-propyl-L-tyrosine, a tri-O-acetyl-GlcNAcB-serine, an L-Dopa, a fluorinated phenylalanine, an isopropyl-L-phenylalanine, a p-acyl-L-phenylalanine, a p-benzoyl-L-phenylalanine, an L-phosphoserine, a phosphono serine, a phosphonotyrosine, a p-iodo-phenylalanine, a p-bromophenylalanine, a p-amino-L-phenylalanine, and an isopropyl-L-phenylalanine. In certain embodiments, the nsAA comprises an O-methyl-L-tyrosine. In certain embodiments, the nsAA comprises an L-3-(2-naphthyl)alanine. In certain embodiments, the O-tRNA recognizes a selector codon. In certain embodiments, the selector codon is an amber codon.

In certain embodiments, the OTS comprises a polynucleotide comprising at least one selector codon that is recognized by the O-tRNA. In certain embodiments, the OTS further comprises a mutant EF-Tu. In certain embodiments, the OTS is a cell-free translation system. In certain embodiments, the cell-free translation system is a cell lysate. In certain embodiments, the cell-free translation system is a reconstituted system.

In certain embodiments, the OTS is a cellular translation system.

In another aspect, this disclosure relates to a cell comprising the OTS as described herein. In certain embodiments, the cell is a non-eukaryotic cell or a prokaryotic cell. In certain embodiments, the prokaryotic cell is Escherichia coli. In certain embodiments, the cell is a eukaryotic cell. In certain embodiments, the cell is a yeast cell. In certain embodiments, the cell is a fungal cell. In certain embodiments, the cell is a mammalian cell. In certain embodiments, the cell is an insect cell. In certain embodiments, the cell is a plant cell. In certain embodiments, the cell encodes a mutation in an EF-Tu. In certain embodiments, the cell has reduced expression of Release Factor 1 compared to an otherwise identical wild-type cell.

In another aspect, this disclosure relates to a polypeptide comprising at least one nsAA, wherein the polypeptide is produced by an OTS or a cell as described herein. In certain embodiments, the polypeptide comprises an antibody or antigen binding fragment thereof. In certain embodiments, the polypeptide comprises human growth hormone.

In certain embodiments, the polynucleotide comprises a nucleic acid sequence encoding an O-tRNA comprising a nucleic acid sequence consisting of a sequence set forth in any one of SEQ ID NOs: 2-31. In certain embodiments, the polynucleotide further comprises a nucleic acid sequence complementary to the O-tRNA sequence consisting of a sequence set forth in any one of SEQ ID NOs: 2-31. In certain embodiments the polynucleotide or set of polynucleotides comprises a nucleic acid sequence encoding an O-RS comprising the amino acid sequence forth in SEQ ID NO: 39. In certain embodiments, the polynucleotide further comprises a nucleic acid sequence complementary to the O-RS-encoding nucleic acid sequence. In certain embodiments, the polynucleotide or set of polynucleotides comprises a nucleic acid sequence of an O-tRNA consisting of the a nucleic acid sequence set forth in any one of SEQ ID NOs: 2-31 and a nucleic acid sequence encoding M. jannaschii tyrosyl-tRNA synthetase. In certain embodiments, the O-RS comprises an amino acid sequence consisting of a sequence set forth in SEQ ID NO: 35 or 39.

In another aspect, described herein is a vector comprising at least one polynucleotide as described herein. In certain embodiments, the vector is an expression vector. In certain embodiments, the vector is selected from the group consisting of a plasmid, a cosmid, a phage, and a virus.

In another aspect, this disclosure relates to a cell comprising a polynucleotide or a vector as described herein.

In another aspect, this disclosure relates to a kit comprising one or more of a polynucleotide(s), a vector, or a cell as described herein, and instructions for use.

In another aspect, this disclosure relates to methods of producing a polypeptide comprising at least one nsAA, comprising expressing in a cell an O-tRNA comprising a nucleic acid sequence at least 85% identical to the sequence set forth in SEQ ID NO: 1 and comprising a deletion of the cytosine located at nucleic acid position 16 of the O-tRNA, wherein the nucleic acid positions correspond to the sequence set forth in SEQ ID NO: 1; and wherein the O-tRNA is capable of being aminoacylated with at least one non-standard amino acid (nsAA) by an O-RS. In certain embodiments, the nucleic acid sequence is at least 90% identical, at least 91% identical, at least 92% identical, at least 93% identical, at least 94% identical, at least 95% identical, at least 96% identical, at least 97% identical, at least 98% identical, or at least 98.7% identical to the sequence set forth in SEQ ID NO: 1. In certain embodiments, the O-tRNA comprises an adenine at nucleic acid position 53 and a uracil at nucleic acid position 63, wherein the nucleic acid positions correspond to the sequence set forth in SEQ ID NO: 1. In certain embodiments, the O-tRNA comprises a nucleic acid sequence consisting of the sequence set forth in SEQ ID NO: 2. In certain embodiments, the O-tRNA comprises a cytosine at nucleic acid positions 3 and 6; a uracil at nucleic acid position 7, an adenosine at nucleic acid position 67, and a guanine at nucleic acid positions 68 and 71, wherein the nucleic acid positions correspond to the sequence set forth in SEQ ID NO: 1. In certain embodiments, the O-tRNA comprises a nucleic acid sequence consisting of the sequence set forth in SEQ ID NO: 3. In certain embodiments, the O-tRNA comprises a nucleic acid sequence set forth in SEQ ID NO: 4. In certain embodiments, the O-tRNA comprises the sequence CAG-AGGGCAG at nucleic acid positions 13 to 23, wherein the nucleic acid positions correspond to the sequence set forth in SEQ ID NO: 1. In another aspect, the disclosure relates to a method of producing a polypeptide comprising at least one nsAA, comprising expressing in a cell an O-tRNA comprising a nucleic acid sequence consisting of a sequence set forth in SEQ ID NO: 36, wherein the sequence does not comprise SEQ ID NO: 1, SEQ ID NO: 37, or SEQ ID NO: 38. In certain embodiments, the O-tRNA comprises a cytosine at position 3. In certain embodiments, the O-tRNA comprises an adenine at position 4. In certain embodiments, the O-tRNA comprises a uracil at position 5. In certain embodiments, the O-tRNA comprises a cytosine at position 6. In certain embodiments, the O-tRNA comprises a uracil at position 7. In certain embodiments, the O-tRNA comprises a guanine at position 46. In certain embodiments, the O-tRNA comprises a uracil at position 48. In certain embodiments, the O-tRNA comprises an adenine at position 50. In certain embodiments, the O-tRNA comprises a guanine at position 51. In certain embodiments, the O-tRNA comprises an adenine at position 53. In certain embodiments, the O-tRNA comprises a uracil at position 63. In certain embodiments, the O-tRNA comprises a cytosine at position 64. In certain embodiments, the O-tRNA comprises a cytosine at position 65. In certain embodiments, the O-tRNA comprises a uracil at position 66. In certain embodiments, the O-tRNA comprises an adenine at position 67. In certain embodiments, the O-tRNA comprises a guanine at position 68. In certain embodiments, the O-tRNA comprises an adenine or a uracil at position 69. In certain embodiments, the O-tRNA comprises a uracil at position 70. In certain embodiments, the O-tRNA comprises a guanine at position 71. In certain embodiments, the O-tRNA comprises a cytosine at position 3, a cytosine at position 6, and a uracil at position 7. In certain embodiments, the O-tRNA comprises a guanine at position 46 and a uracil at position 48. In certain embodiments, the O-tRNA comprises an adenine at position 67, a guanine at position 68, and a guanine at position 71.

In certain embodiments, the O-tRNA comprises a nucleic acid sequence consisting of the sequence set forth in any one of SEQ ID NOs: 2-16.

In certain embodiments, the method further comprises expressing an O-RS in the cell. In certain embodiments, the O-RS comprises an O-RS of an M. jannaschii tyrosyl-tRNA synthetase. In certain embodiments, the O-RS comprises an amino acid sequence consisting of the sequence set forth in SEQ ID NO: 35 or 39. In certain embodiments, the O-tRNA comprises a nucleic acid sequence set forth in any one of SEQ ID NOs: 2-16 and the O-RS comprises an amino acid sequence consisting of the sequence set forth in SEQ ID NO: 35 or 39.

In certain embodiments of the method, the O-RS aminoacylates the O-tRNA with the nsAA. In certain embodiments, the nsAA has the structure according to Formula I; and wherein the R group is any substituent other than a corresponding substituent used in the twenty natural amino acids. In certain embodiments, the nsAA has the structure according to Formula I; and wherein the R group comprises an alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amine, or combinations thereof. In certain embodiments, the nsAA is selected from the group consisting of an amino acid comprising a photoactivatable cross-linker, a spin-labeled amino acid, a fluorescent amino acid, a metal binding amino acid, a metal containing amino acid, a radioactive amino acid, an amino acid with at least one novel functional group, an amino acid that covalently or noncovalently interacts with other molecules, a photocaged amino acid, a photoisomerizable amino acid, an amino acid comprising biotin or a biotin analogue, a carbohydrate-modified amino acid, and an amino acid comprising polyethylene glycol or polyether, a heavy atom substituted amino acid, a chemically cleavable amino acid, a photocleavable amino acid, and combinations thereof. In certain embodiments, the nsAA comprises a tyrosine analog. In certain embodiments, the tyrosine analog is selected from the group consisting of a para-substituted tyrosine, an ortho-substituted tyrosine, and a meta-substituted tyrosine. In certain embodiments, the substituted tyrosine comprises a keto group, an acetyl group, a benzoyl group, an amino group, a hydrazine, a hydroxyamine, a thiol group, a carboxy group, an isopropyl group, a methyl group, a branched hydrocarbon, a saturated or unsaturated hydrocarbon, an O-methyl group, a polyether group, a nitro group, or combinations thereof. In certain embodiments, the nsAA comprises a glutamine analog. In certain embodiments, the glutamine analog comprises a α-hydroxy derivative, a T-substituted derivative, a cyclic derivative, or an amide substituted glutamine derivative. In certain embodiments, the nsAA comprises a phenylalanine analog. In certain embodiments, the phenylalanine analog is an amino-, an isopropyl-, or a O-allyl-containing phenylalanine analog. In certain embodiments, the phenylalanine analog is selected from the group consisting of a para-substituted phenylalanine, an ortho-substituted phenylalanine, and a meta-substituted phenylalanine. In certain embodiments, the substituent comprises a hydroxy group, a methoxy group, a methyl group, an allyl group, an aldehyde, an azido, an iodo, a bromo, a keto group or an acetyl group. In certain embodiments, the nsAA comprises a para-acetyl phenylalanine.

In certain embodiments of the method, the nsAA is selected from the group consisting of a p-propargyl phenylalanine, a O-methyl-L-tyrosine, an L-3-(2-naphthyl)alanine, a 3-methylphenylalanine, an O-4-allyl-L-tyrosine, a 4-propyl-L-tyrosine, a tri-O-acetyl-GlcNAcB-serine, an L-Dopa, a fluorinated phenylalanine, an isopropyl-L-phenylalanine, a p-acyl-L-phenylalanine, a p-benzoyl-L-phenylalanine, an L-phosphoserine, a phosphono serine, a phosphonotyrosine, a p-iodo-phenylalanine, a p-bromophenylalanine, a p-amino-L-phenylalanine, and an isopropyl-L-phenylalanine.

In certain embodiments of the method, the nsAA is selected from the group consisting of 4-acetyl-phenylalanine (AcF), 4-azido-phenylalanine (AzF); 4-propargyloxyphenylalanine (PaF); and 4-aminophenylalanine (AmF). In certain embodiments, the nsAA comprises an 4-acetyl-phenylalanine (AcF). In certain embodiments, the nsAA comprises an 4-azido-phenylalanine (AzF). In certain embodiments, In certain embodiments, the nsAA comprises an 4-propargyloxyphenylalanine (PaF). In certain embodiments, the nsAA comprises an 4-aminophenylalanine (AmF). In certain embodiments, the nsAA is biosynthesized by the cell. In certain embodiments, the nsAA is provided to the cell exogenously.

In certain embodiments of the method, the cell is a non-eukaryotic cell or a prokaryotic cell. In certain embodiments, the prokaryotic cell is Escherichia coli. In certain embodiments, the cell is a eukaryotic cell. In certain embodiments, the eukaryotic cell is a yeast cell. In certain embodiments, the eukaryotic cell is a fungal cell. In certain embodiments, eukaryotic cell is a mammalian cell. In certain embodiments, the eukaryotic cell is an insect cell. In certain embodiments, the eukaryotic cell is a plant cell.

In certain embodiments of the method, the O-tRNA recognizes a selector codon. In certain embodiments, the selector codon is an amber codon. In certain embodiments, the polypeptide comprises an antibody or antigen binding fragment thereof. In certain embodiments, the polypeptide comprises human growth hormone. In certain aspects, disclosed herein is a method of producing a polypeptide comprising at least one nsAA, comprising providing: i) an O-tRNA comprising a nucleic acid sequence at least 85% identical to the sequence set forth in SEQ ID NO: 1 and comprising a deletion of the cytosine located at nucleic acid position 16 of the O-tRNA; wherein the nucleic acid positions correspond to the sequence set forth in SEQ ID NO: 1; and wherein the O-tRNA is capable of being aminoacylated with at least one non-standard amino acid (nsAA) by an O-RS; ii) an O-RS; wherein the O-RS aminoacylates the O-tRNA with the nsAA; and iii) a polynucleotide encoding the polypeptide, wherein the polynucleotide comprises at least one selector codon; and wherein the O-tRNA recognizes the selector codon. In certain embodiments, wherein the nucleic acid sequence is at least 90% identical, at least 91% identical, at least 92% identical, at least 93% identical, at least 94% identical, at least 95% identical, at least 96% identical, at least 97% identical, at least 98% identical, or at least 98.7% identical to the sequence set forth in SEQ ID NO: 1. In certain embodiments, the O-tRNA comprises an adenine at nucleic acid position 53 and a uracil at nucleic acid position 63, wherein the nucleic acid positions correspond to the sequence set forth in SEQ ID NO:1. In certain embodiments, the O-tRNA comprises a nucleic acid sequence consisting of a sequence set forth in SEQ ID NO: 2. In certain embodiments, the O-tRNA comprises a cytosine at nucleic acid positions 3 and 6; a uracil at nucleic acid position 7, an adenosine at nucleic acid position 67, and a guanine at nucleic acid positions 68 and 71, wherein the nucleic acid positions correspond to the sequence set forth in SEQ ID NO: 1. In certain embodiments, the O-tRNA comprises a nucleic acid sequence consisting of a sequence set forth in SEQ ID NO: 3. In certain embodiments, the O-tRNA comprises a nucleic acid sequence consisting of a sequence set forth in SEQ ID NO: 4. In certain embodiments, the O-tRNA comprises the sequence CAG-AGGGCAG at nucleic acid positions 13 to 23, wherein the nucleic acid positions correspond to the sequence set forth in SEQ ID NO: 1.

In another aspect, the disclosure relates to a method of producing a polypeptide comprising at least one non-standard amino acid (nsAA), the method comprising i) an O-tRNA comprising a nucleic acid sequence consisting of a sequence set forth in SEQ ID NO: 36, wherein the sequence does not comprise SEQ ID NO: 1, SEQ ID NO: 37, or SEQ ID NO: 38; ii) an O-RS; wherein the O-RS aminoacylates the O-tRNA with the nsAA; and iii) a polynucleotide encoding the polypeptide, wherein the polynucleotide comprises at least one selector codon; and wherein the O-tRNA recognizes the selector codon. In certain embodiments, the O-tRNA comprises a cytosine at position 3. In certain embodiments, the 0-tRNA comprises an adenine at position 4. In certain embodiments, the O-tRNA comprises a uracil at position 5. In certain embodiments, the O-tRNA comprises a cytosine at position 6. In certain embodiments, the O-tRNA comprises a uracil at position 7. In certain embodiments, the O-tRNA comprises a guanine at position 46. In certain embodiments, the O-tRNA comprises a uracil at position 48. In certain embodiments, the O-tRNA comprises an adenine at position 50. In certain embodiments, the O-tRNA comprises a guanine at position 51. In certain embodiments, the O-tRNA comprises an adenine at position 53. In certain embodiments, the O-tRNA comprises a uracil at position 63. In certain embodiments, the O-tRNA comprises a cytosine at position 64. In certain embodiments, the O-tRNA comprises a cytosine at position 65. In certain embodiments, the O-tRNA comprises a uracil at position 66. In certain embodiments, the O-tRNA comprises an adenine at position 67. In certain embodiments, the O-tRNA comprises a guanine at position 68. In certain embodiments, the O-tRNA comprises an adenine or a uracil at position 69. In certain embodiments, the O-tRNA comprises a uracil at position 70. In certain embodiments, the O-tRNA comprises a guanine at position 71. In certain embodiments, the O-tRNA comprises a cytosine at position 3, a cytosine at position 6, and a uracil at position 7. In certain embodiments, the O-tRNA comprises a guanine at position 46 and a uracil at position 48. In certain embodiments, the O-tRNA comprises an adenine at position 67, a guanine at position 68, and a guanine at position 71.

In certain embodiments, the O-tRNA comprises a nucleic acid sequence consisting of the set forth in any one of SEQ ID NOs: 2-16. In certain embodiments, the O-RS comprises an O-RS of an M. jannaschii tyrosyl-tRNA synthetase. In certain embodiments, the O-RS comprises an amino acid sequence consisting of the sequence set forth in SEQ ID NO: 35 or 39.

In certain embodiments of the method, the nsAA has the structure according to Formula I; and wherein the R group is any substituent other than a corresponding substituent used in the twenty natural amino acids. In certain embodiments, the nsAA has the structure according to Formula I; and wherein the R group comprises an alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amine, or combinations thereof. In certain embodiments, the nsAA is selected from the group consisting of an amino acid comprising a photoactivatable cross-linker, a spin-labeled amino acid, a fluorescent amino acid, a metal binding amino acid, a metal containing amino acid, a radioactive amino acid, an amino acid with at least one novel functional group, an amino acid that covalently or noncovalently interacts with other molecules, a photocaged amino acid, a photoisomerizable amino acid, an amino acid comprising biotin or a biotin analogue, a carbohydrate-modified amino acid, an amino acid comprising polyethylene glycol or polyether, a heavy atom substituted amino acid, a chemically cleavable amino acid, a photocleavable amino acid, and combinations thereof. In certain embodiments, the nsAA comprises a tyrosine analog. In certain embodiments, the tyrosine analog is selected from the group consisting of a para-substituted tyrosine, an ortho-substituted tyrosine, and a meta-substituted tyrosine. In certain embodiments, the substituted tyrosine comprises a keto group, an acetyl group, a benzoyl group, an amino group, a hydrazine, a hydroxyamine, a thiol group, a carboxy group, an isopropyl group, a methyl group, a branched hydrocarbon, a saturated or unsaturated hydrocarbon, an O-methyl group, a polyether group, a nitro group, or combinations thereof. In certain embodiments, the nsAA comprises a glutamine analog. In certain embodiments, the glutamine analog comprises a α-hydroxy derivative, a T-substituted derivative, a cyclic derivative, an amide substituted glutamine derivative. In certain embodiments, the nsAA comprises a phenylalanine analog. In certain embodiments, the phenylalanine analog is an amino-, an isopropyl-, or an O-allyl-containing phenylalanine analog. In certain embodiments, the phenylalanine analog is selected from the group consisting of a para-substituted phenylalanine, an ortho-substituted phenylalanine, and a meta-substituted phenylalanine. In certain embodiments, the substituent comprises a hydroxy group, a methoxy group, a methyl group, an allyl group, an aldehyde, an azido, an iodo, a bromo, a keto group or an acetyl group. In certain embodiments, the nsAA comprises a para-acetyl phenylalanine.

In certain embodiments, the nsAA is selected from the group consisting of a p-propargyl phenylalanine, an O-methyl-L-tyrosine, an L-3-(2-naphthyl)alanine, a 3-methylphenylalanine, an O-4-allyl-L-tyrosine, a 4-propyl-L-tyrosine, a tri-O-acetyl-GlcNAcB-serine, an L-Dopa, a fluorinated phenylalanine, an isopropyl-L-phenylalanine, a p-acyl-L-phenylalanine, a p-benzoyl-L-phenylalanine, an L-phosphoserine, a phosphono serine, a phosphonotyrosine, a p-iodo-phenylalanine, a p-bromophenylalanine, a p-amino-L-phenylalanine, and an isopropyl-L-phenylalanine. In certain embodiments, the nsAA is selected from the group consisting of 4-acetyl-phenylalanine (AcF), 4-azido-phenylalanine (AzF); 4-propargyloxyphenylalanine (PaF); and 4-aminophenylalanine (AmF). In certain embodiments, the nsAA comprises a 4-acetyl-phenylalanine (AcF). In certain embodiments, the nsAA comprises a 4-azido-phenylalanine (AzF). In certain embodiments, the nsAA comprises a 4-propargyloxyphenylalanine (PaF). In certain embodiments, the nsAA comprises a 4-aminophenylalanine (AmF).

In certain embodiments of the methods, the selector codon is an amber codon. In certain embodiments, the polypeptide comprises an antibody, an antigen binding fragment, or component thereof, e.g., an antibody heavy chain variable domain, an antibody light chain variable domain, an antibody heavy chain, an antibody light chain, or a scFV. In certain embodiments, the polypeptide comprises human growth hormone. In certain embodiments, the polypeptide is produced by a cell-free translation system. In certain embodiments, the cell-free translation system is a cell lysate. In certain embodiments, the cell-free translation system is a reconstituted system.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 is an illustration of a plasmid map for exemplary constructs containing candidate OTSs according to the disclosure.

FIG. 2 is an image of a western blot after ProA/PhyTip purification of Trastuzumab with nsAA incorporation at various strategic sites produced under shake-flask fermentation conditions.

FIG. 3 is an image of results from SDS-PAGE after AKTA™ purification of Trastuzumab with substitution of S123 for p-Acetylphenylalanine produced under shake-flask fermentation conditions.

FIG. 4 are graphs showing the evaluation of fidelity (average maximum misincorporation frequency “MMF”) and efficiency (average read through efficiency “RRE”) of the MG72, MG33 and MG24 candidate OTSs compared to the prior known F12, F13 and F14 OTSs using the RFP-GFP fusion protein reporter. Average OD is also shown.

FIG. 5 is a diagram of the tRNA cloverleaf structures and sequences of the MG72, MG33 and MG24 and F12 tRNAs.

FIG. 6 is a diagram of additional tRNA cloverleaf structures and sequences of candidate tRNAs disclosed herein.

FIG. 7 is a diagram showing sequence alignments of the candidate tRNAs and F12, F13 and F14 tRNAs disclosed herein.

### DETAILED DESCRIPTION

### Definitions

Terms used in the claims and specification are defined as set forth below unless otherwise specified.

As used herein, the term “orthogonal” refers to a molecule (e.g., an orthogonal tRNA (O-tRNA) and/or an orthogonal aminoacyl tRNA synthetase (0-RS)) that is used with reduced efficiency by a system of interest (e.g., a translational system, e.g., a cell) or that fails to function with endogenous components of the cell. In the context of tRNAs and aminoacyl-tRNA synthetases, orthogonal refers to the inability or reduced efficiency, e.g., less than 20% efficient, less than 10% efficient, less than 5% efficient, or e.g., less than 1% efficient, of an orthogonal tRNA and/or orthogonal RS to function in the translation system of interest. The orthogonal molecule lacks a functional endogenous complementary molecule in the cell. For example, an orthogonal tRNA in a translation system of interest is aminoacylated by any endogenous RS of a translation system of interest with reduced or even zero efficiency, when compared to aminoacylation of an endogenous tRNA by an endogenous RS. In another example, an orthogonal RS aminoacylates any endogenous tRNA in the translation system of interest with reduced or even zero efficiency, as compared to aminoacylation of the endogenous tRNA by an endogenous RS. A second orthogonal molecule can be introduced into the cell that functions with the first orthogonal molecule. For example, an orthogonal tRNA/RS pair includes introduced complementary components that function together in the cell with an efficiency (e.g., about 50% efficiency, about 60% efficiency, about 70% efficiency, about 75% efficiency, about 80% efficiency, about 85% efficiency, about 90% efficiency, about 95% efficiency, or about 99% or more efficiency) to that of a tRNA/RS standard amino acid pair.

The term “cognate” refers to components that function together, e.g., a tRNA and an aminoacyl-tRNA synthetase. The components can also be referred to as being complementary.

The term “aminoacylates” refers to transferring of an amino acid to a tRNA by an amino-acyl tRNA synthetase.

The term “preferentially aminoacylates” refers to an efficiency, e.g., about 70% efficient, about 75% efficient, about 80% efficient, about 85% efficient, about 90% efficient, about 95% efficient, or about 99% or more efficient, at which an O-RS aminoacylates an O-tRNA with a selected amino acid, e.g., an nsAA, compared to the O-RS aminoacylating a naturally occurring tRNA or a starting material used to generate the O-tRNA. The nsAA is then incorporated into a growing polypeptide chain with high fidelity, e.g., at greater than about 70% fidelity, at greater than about 75% fidelity, at greater than about 80% fidelity, at greater than about 85% fidelity, at greater than about 90% fidelity, greater than about 95% fidelity, or greater than about 99% fidelity.

The term “selector codon” refers to codons recognized by the O-tRNA in the translation process and not recognized by an endogenous tRNA. The O-tRNA anti codon loop recognizes the selector codon on the mRNA and incorporates its non-standard amino acid (nsAA), at this site in the polypeptide. Selector codons can include but are not limited to, e.g., nonsense codons, such as, stop codons, including but not limited to, amber, ochre, and opal codons; four or more base codons; rare codons; codons derived from natural or unnatural base pairs and/or the like. For a given system, a selector codon can also include one of the natural three base codons, wherein the endogenous system does not use (or rarely uses) said natural three base codon. For example, this includes a system that is lacking a tRNA that recognizes the natural three base codon, and/or a system wherein the natural three base codon is a rare codon.

The term “non-standard amino acid” (nsAA) refers to any amino acid not naturally occurring in proteins (e.g., a non-naturally occurring modified amino acid or amino acid analogue). In other words, the non-standard amino acid are amino acids other than selenocysteine and/or pyrrolysine and the following twenty genetically encoded alpha-amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

Abbreviations used in this application include the following: non-standard amino acid (nsAA), transfer RNA (tRNA), orthogonal tRNA (O-tRNA), and orthogonal amino acyl tRNA synthetase (0-RS).

The term “translation system” refers to the components necessary to incorporate a naturally occurring amino acid into a growing polypeptide chain (protein). Components of a translation system can include, e.g., ribosomes, tRNA's, synthetases, mRNA and the like. The components of the present disclosure can be added to an in vitro or in vivo translation system. Examples of translation systems include but are not limited to, a non-eukaryotic cell, e.g., a bacterium (such as E. coli), a eukaryotic cell, e.g., a yeast cell, a mammalian cell, a plant cell, an algae cell, a fungus cell, an insect cell, a cell-free translational system e.g., a cell lysate, and/or the like.

The term percent “identity,” in the context of two or more polypeptide or nucleic acid sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

It must be noted that, as used in the specification and the appended claims, the singular forms “a,” “an” and “the” include plural referents unless the context clearly dictates otherwise.

This disclosure provides for tRNAs and the corresponding aminoacyl tRNA synthetases for efficient production of proteins containing non-standard amino acids. These engineered orthogonal tRNA (O-tRNA)/orthogonal aminoacyl tRNA synthetase (O-RS) pair, i.e., Orthogonal Translation Systems (OTSs), can be used to incorporate an nsAA in a specific position in a growing polypeptide in response to a selector codon that is recognized by the tRNA. This disclosure provides for Orthogonal Translation Systems (OTSs) with superior fidelity and efficiency in nsAA incorporation compared to known systems.

Orthogonal tRNA (O-tRNA)

Described herein are orthogonal transfer RNAs (O-tRNAs) that are aminoacylated with a non-standard amino acid (nsAA). The O-tRNA mediates incorporation of an nsAA into a protein that is encoded by a polynucleotide that comprises a selector codon that is recognized by the O-tRNA.

In certain aspects, described herein are orthogonal tRNAs (O-tRNA) comprising a nucleic acid sequence at least 85% identical to the sequence set forth in SEQ ID NO: 1 and comprising a deletion of the cytosine located at nucleic acid position 16 of the O-tRNA, wherein the nucleic acid positions correspond to the sequence set forth in SEQ ID NO: 1; and wherein the O-tRNA is capable of being aminoacylated with at least one non-standard amino acid (nsAA) by an orthogonal aminoacyl tRNA synthetase (O-RS). In certain embodiments, the nucleic acid sequence is at least 90% identical, at least 91% identical, at least 92% identical, at least 93% identical, at least 94% identical, at least 95% identical, at least 96% identical, at least 97% identical, at least 98% identical, or at least 98.7% identical to the sequence set forth in SEQ ID NO: 1. In certain embodiments, the O-tRNA comprises an adenine at nucleic acid position 53 and a uracil at nucleic acid position 63, wherein the nucleic acid positions correspond to the sequence set forth in SEQ ID NO: 1. In certain embodiments, the O-tRNA comprises a nucleic acid sequence set forth in SEQ ID NO: 2. In certain embodiments, the O-tRNA comprises a cytosine at amino acid positions 3 and 6; a uracil at nucleic acid position 7, an adenosine at nucleic acid position 67, and a guanine at nucleic acid positions 68 and 71, wherein the nucleic acid positions correspond to the sequence set forth in SEQ ID NO: 1. In certain embodiments, the O-tRNA comprises a nucleic acid sequence consisting of the sequence set forth in SEQ ID NO: 3. In certain embodiments, the O-tRNA comprises a nucleic acid sequence consisting of the sequence set forth in SEQ ID NO: 4. In certain embodiments, the O-tRNA comprises the sequence CAG-AGGGCAG at nucleic acid positions 13 to 23, wherein the nucleic acid positions correspond to the sequence set forth in SEQ ID NO: 1.

In certain aspects, the disclosure relates to an O-tRNA comprising a nucleic acid sequence consisting of a sequence set forth in SEQ ID NO: 36, wherein the sequence does not comprise SEQ ID NO: 1, SEQ ID NO: 37, or SEQ ID NO: 38.

SEQ ID NO: 36 provides a consensus sequence as follows: CCX₁X₂X₃X₄X₅UAGUUCAGX₆AGGGCAGAACGGCGGACUCUAAAUCCGCAX₇GX₈C X₉X₁₀X₁₁X₁₂GUCAAAUCX₁₃X₁₄X₁₅X₁₆X₁₇X₁₈X₁₉X₂₀X₂₁GGACCA; wherein X₁ is C or G; X₂ is A or G; X₃ is A, U or C; X₄ is C or G; X₅ is U or G; X₆ is C or del; X₇ is G or U; X₈ is G or U; X₉ is A or G; X₁₀ is G or C; X₁₁ is G, C, A or U; X₁₂ is G or A; X₁₃ is C or U; X₁₄ is G, C, A or U; X₁₅ is G or C; X₁₆ is U or C; X₁₇ is A or C; X₁₈ is G or C; X₁₉ is A, G or U; X₂₀ is U or C; and X₂₁ is C or G.

In certain embodiments, the O-tRNA comprises a cytosine at position 3. In certain embodiments, the O-tRNA comprises an adenine at position 4. In certain embodiments, the O-tRNA comprises a uracil at position 5. In certain embodiments, the O-tRNA comprises a cytosine at position 6. In certain embodiments, the O-tRNA comprises a uracil at position 7. In certain embodiments, the O-tRNA comprises a guanine at position 46. In certain embodiments, the O-tRNA comprises a uracil at position 48. In certain embodiments, the O-tRNA comprises an adenine at position 50. In certain embodiments, the O-tRNA comprises a guanine at position 51. In certain embodiments, the O-tRNA comprises an adenine at position 53. In certain embodiments, the O-tRNA comprises a uracil at position 63. In certain embodiments, the O-tRNA comprises a cytosine at position 64. In certain embodiments, the O-tRNA comprises a cytosine at position 65. In certain embodiments, the O-tRNA comprises a uracil at position 66. In certain embodiments, the O-tRNA comprises an adenine at position 67. In certain embodiments, the O-tRNA comprises a guanine at position 68. In certain embodiments, the O-tRNA comprises an adenine or a uracil at position 69. In certain embodiments, the O-tRNA comprises a uracil at position 70. In certain embodiments, the O-tRNA comprises a guanine at position 71. In certain embodiments, the O-tRNA comprises a cytosine at position 3, a cytosine at position 6, and a uracil at position 7. In certain embodiments, the O-tRNA comprises a guanine at position 46 and a uracil at position 48. In certain embodiments, the O-tRNA comprises an adenine at position 67, a guanine at position 68, and a guanine at position 71.

In certain embodiments, the O-tRNA comprises an adenine at nucleic acid position 52 and a uracil at nucleic acid position 62; wherein the nucleic acid positions correspond to the sequence set forth in SEQ ID NO: 1. In certain embodiments, the O-tRNA comprises an guanine at nucleic acid position 52 and a cytosine at nucleic acid position 62; wherein the nucleic acid positions correspond to the sequence set forth in SEQ ID NO: 1. In certain embodiments, the O-tRNA comprises an guanine at nucleic acid position 51 and a cytosine at nucleic acid position 65; wherein the nucleic acid positions correspond to the sequence set forth in SEQ ID NO: 1. In certain embodiments, the O-tRNA comprises an cytosine at nucleic acid positions 3 and 6, a uracil at nucleic acid position 7, an adenine at nucleic acid position 66, and guanines at nucleic acid positions 67 and 70; wherein the nucleic acid positions correspond to the sequence set forth in SEQ ID NO: 1. In certain embodiments, the O-tRNA comprises a uracil at nucleic acid positions 5, and 7, an adenine at nucleic acid positions 67 and 69; wherein the nucleic acid positions correspond to the sequence set forth in SEQ ID NO: 1. In certain embodiments, the O-tRNA comprises an adenine at nucleic acid positions 4 and 67, a uracil at nucleic acid positions 7 and 70, an cytosine at nucleic acid position 6 and a guanine at nucleic acid position 68; wherein the nucleic acid positions correspond to the sequence set forth in SEQ ID NO: 1. In certain embodiments, the O-tRNA comprises an adenine at nucleic acid position 5, and a uracil at nucleic acid positions 69 and 70; wherein the nucleic acid positions correspond to the sequence set forth in SEQ ID NO: 1. In certain embodiments, the O-tRNA comprises a uracil at nucleic acid position 48; wherein the nucleic acid positions correspond to the sequence set forth in SEQ ID NO: 1. In certain embodiments, the O-tRNA comprises a guanine at nucleic acid position 51; wherein the nucleic acid positions correspond to the sequence set forth in SEQ ID NO: 1. In certain embodiments, the O-tRNA comprises a guanine at nucleic acid position 46 and a uracil at nucleic acid position 48; wherein the nucleic acid positions correspond to the sequence set forth in SEQ ID NO: 1. In certain embodiments, the O-tRNA comprises a guanine at nucleic acid position 51 and a uracil at nucleic acid position 48; wherein the nucleic acid positions correspond to the sequence set forth in SEQ ID NO: 1. In certain embodiments, the O-tRNA comprises a guanine at nucleic acid positions 46 and 51 and a uracil at nucleic acid position 48; wherein the nucleic acid positions correspond to the sequence set forth in SEQ ID NO: 1.

In certain embodiments, the O-tRNAs disclosed herein comprise one or more mutations compared to the wild-type M. jannaschii tyrosyl-tRNA (SEQ ID NO: 32); or one or more mutations compared to the F12 O-tRNA sequence (SEQ ID NO: 1), the F13 O-tRNA sequence (SEQ ID NO: 37), or the F14 O-tRNA sequence (SEQ ID NO: 38). For example, in certain embodiments, nucleotides residing in the stem of the T loop of the O-tRNA are mutated, e.g. G53 and C63 are mutated to A52 and U62; wherein the nucleic acid positions correspond to the sequence set forth in SEQ ID NO: 1. In certain embodiments, paired nucleotides residing in the stem of the T loop of the O-tRNA, i.e., C52 and G64, or U52 and A64, or A52 and U64 are mutated to G52 and C62; wherein the nucleic acid positions correspond to the sequence set forth in SEQ ID NO: 1. In certain embodiments, nucleotides residing in the stem of the T loop of the O-tRNA, i.e., C51 and G65, are mutated to G51 and C65; wherein the nucleic acid positions correspond to the sequence set forth in SEQ ID NO: 1. In certain embodiments, nucleotides residing in the stem of the acceptor stem of the O-tRNA, i.e., G3, G6, G7, C67, C68 and C71 are mutated to C3, C6, U7, A66, G67 and G70; wherein the nucleic acid positions correspond to the sequence set forth in SEQ ID NO: 1. In certain embodiments, nucleotides residing in the stem of the acceptor stem of the O-tRNA, i.e., C5, G7, C67 and G69 are mutated to U5, U7, A67 and A69; wherein the nucleic acid positions correspond to the sequence set forth in SEQ ID NO: 1. In certain embodiments, nucleotides residing in the stem of the acceptor stem of the O-tRNA, i.e., G4, G6, G7, C67, C68 and C70 are mutated to A4, C6, U7, A67, G68 and U70; wherein the nucleic acid positions correspond to the sequence set forth in SEQ ID NO: 1. In certain embodiments, nucleotides residing in the stem of the acceptor stem of the O-tRNA, i.e., C5, G69 and C70 are mutated to A5, U69 and U70; wherein the nucleic acid positions correspond to the sequence set forth in SEQ ID NO: 1. In certain embodiments, a nucleotide residing in the variable loop of the O-tRNA, i.e., G48 is mutated to U48; wherein the nucleic acid positions correspond to the sequence set forth in SEQ ID NO: 1. In certain embodiments, a nucleotide residing in the variable loop, i.e., C51 is mutated to G51; wherein the nucleic acid positions correspond to the sequence set forth in SEQ ID NO: 1. In certain embodiments, a nucleotide residing in the variable loop, i.e., U46, and G48 are mutated to G46 and U48; wherein the nucleic acid positions correspond to the sequence set forth in SEQ ID NO: 1. In certain embodiments, a nucleotides residing in the variable loop, i.e., G48 and C51 are mutated to U48 and G51; wherein the nucleic acid positions correspond to the sequence set forth in SEQ ID NO: 1. In certain embodiments, a nucleotide residing in the variable loop, i.e., U46, G48, and C51 are mutated to G46, U48, and G51.

In certain embodiments, the O-tRNA comprises a nucleic acid sequence consisting of the sequence set forth in any one of SEQ ID NOs: 2-16.

A tRNA may be aminoacylated with a desired amino acid by any method or technique, including but not limited to, chemical or enzymatic aminoacylation. Aminoacylation may be accomplished by aminoacyl tRNA synthetases or by other enzymatic molecules, including but not limited to, ribozymes. The term “ribozyme” is interchangeable with “catalytic RNA.” Thus, in certain embodiments, the O-tRNA is chemically aminoacylated. In certain embodiments, the O-tRNA is enzymatically aminoacylated. In certain embodiments, the O-tRNA is enzymatically aminoacylated by a ribozyme.

The O-tRNAs described herein can be derived from a variety of organisms, e.g., non-vertebrate organisms, such as a prokaryotic organism (e.g., E. coli, Bacillus stearothermophilus, or the like), or an archaebacterium, or e.g., a vertebrate organism. In certain embodiments, the O-tRNA is derived from an archaeal tRNA. In certain embodiments, the O-tRNA is derived from a Methanococcus jannaschii tRNA.

In an aspect, the O-tRNA has an anticodon that will pair with a selector codon. In certain embodiments, the selector codon is the amber stop codon (TAG), thus allowing the incorporation of the nsAA at the TAG codon. Because the TAG codon naturally functions as a stop codon through recognition by Release Factor I (which terminates protein synthesis), competition ensues between incorporation of the nsAA and termination of protein synthesis.

The functionality of OTSs can be further improved by knocking out or reducing the function of the Release Factor I that competes with nsAA incorporation at the amber codon, engineering a protein elongation factor to better accommodate the OTS tRNA, and new methods for OTS directed evolution. Accordingly, in certain embodiments the OTS has reduced expression of Release Factor 1 (e.g., about 15-50% less, about 25-75% less, about 50-100% less or about 75-100% less) compared to an otherwise identical wild-type cell. In certain embodiments, the OTS has no Release Factor I.

In certain embodiments, the O-tRNA is post-transcriptionally modified when expressed in a cell.

Orthogonal Amino-Acyl tRNA Synthetases (O-RS)

Described herein are orthogonal aminoacyl-tRNA synthetases (O-RS) that aminoacylate orthogonal tRNAs with nsAAs. In certain embodiments, the O-RS is derived from Methanococcus jannaschii tyrosyl-tRNA synthetase. In certain embodiments, the O-RS is encoded by the amino acid sequence set forth in SEQ ID NO: 35 or 39. In certain embodiments, the O-RS comprises an amino acid sequence consisting of the sequence set forth in SEQ ID NO: 35 or 39.

In certain embodiments, the O-RS preferentially aminoacylates the O-tRNAs with nsAAs. The term “preferentially aminoacylates” refers to an efficiency, e.g., about 70% efficient, about 75% efficient, about 80% efficient, about 85% efficient, about 90% efficient, about 95% efficient, or about 99% or more efficient, at which an O-RS aminoacylates an O-tRNA with a selected amino acid, e.g., an nsAA, compared to the O-RS aminoacylating a naturally occurring tRNA. In certain embodiments, efficiency is determined by average read through efficiency “RRE”. In certain embodiments, the relative readthrough efficiency (RRE) of the TAG codon is the GFP/RFP fluorescence ratio for the mcherryTAG assay plasmid divided by the GFP/RFP fluorescence ratio for the mcherryTAC control plasmid.

In certain embodiments, the nsAA is then incorporated into a growing polypeptide chain with high fidelity, e.g., at greater than about 70% fidelity for a given selector codon, at greater than about 75% fidelity for a given selector codon, at greater than about 80% fidelity for a given selector codon, at greater than about 85% fidelity for a given selector codon, at greater than about 90% fidelity for a given selector codon, greater than about 95% fidelity for a given selector codon, or greater than about 99% fidelity for a given selector codon. In certain embodiments, fidelity is determined by average maximum misincorporation frequency “MMF”. In certain embodiments, the maximum misincorporation frequency (MMF), is calculated by dividing the RRE when nsAA was not added to the growth media by the RRE when nsAA is present.

The O-RSs described herein can be derived from a variety of organisms, e.g., non-vertebrate organisms, such as a prokaryotic organism (e.g., E. coli, Bacillus stearothermophilus, or the like), or an archaebacterium, or e.g., a vertebrate organism. In certain embodiments, the O-RS is derived from archaea Methanococcus jannaschii.

In certain embodiments, the O-RS has one or more improved or enhanced enzymatic properties for the nsAAs as compared to a natural amino acid. For example, the improved or enhanced properties for the nsAA as compared to a natural amino acid include any of e.g., a higher Km, a lower Km, a higher kcat, a lower kcat, a lower kcat/km, a higher kcat/km, etc.

### Orthogonal Translation Systems (OTSs)

This disclosure describes orthogonal translation systems (OTSs) that comprise an orthogonal aminoacyl-tRNA synthetase (O-RS) and an orthogonal tRNA (O-tRNA) described herein. In certain embodiments, the OTS further comprises an nsAA described herein. Optionally, an nsAA is provided exogenously to the OTS. Alternately, e.g., where the OTS is a cell, the non-standard amino acid can be biosynthesized by the OTS. In certain embodiments, the OTS further comprises a mutant EF-Tu. In certain embodiments, Release Factor I has been removed or modified, or Release Factor I expression has been reduced in the OTS. In certain embodiments, the OTS comprises an engineered or modified protein elongation factor to better accommodate the OTS tRNA during translation (e.g., increase efficacy and/or fidelity). In certain embodiments, the modified elongation factor is an EF-Tu as described in Haruna K. et al. Nucleic Acids Research, Vol 42, Issue 15, 2 Sep. 2014, 9976-9983.

The individual components of an O-tRNA/O-RS pair can be derived from the same organism or different organisms. In an embodiment, the O-tRNA/O-RS pair is from the same organism. Alternatively, the O-tRNA and the O-RS of the O-tRNA/O-RS pair are from different organisms. In certain embodiments, the O-tRNA and the O-RS are derived from archaea. In certain embodiments, the O-tRNA and the O-RS are derived from M. jannaschii.

In certain embodiments, the disclosure provides a cell comprising an orthogonal aminoacyl-tRNA synthetase (O-RS), an orthogonal tRNA (O-tRNA), a nucleic acid that comprises a polynucleotide that encodes a polypeptide of interest, and optionally a nsAA described herein. In an aspect, the polynucleotide comprises at least one selector codon that is recognized by the O-tRNA. In an aspect, the O-RS preferentially aminoacylates the orthogonal tRNA (O-tRNA) with the nsAA in the cell, and the cell produces the polypeptide of interest in the absence of the nsAA, with a yield that is, e.g., less than 30%, less than 20%, less than 15%, less than 10%, less than 5%, less than 2.5%, less than 1%, etc., of the yield of the polypeptide in the presence of the nsAA.

Translation systems may be cellular or cell-free, and may be prokaryotic or eukaryotic. Cellular translation systems include, but are not limited to, whole cell preparations such as permeabilized cells or cell cultures wherein a desired nucleic acid sequence can be transcribed to mRNA and the mRNA translated. Cell-free translation systems are commercially available and many different types and systems are well known.

In certain embodiments, this disclosure provides a cell-free OTS comprising an orthogonal aminoacyl-tRNA synthetase (0-RS), an orthogonal tRNA (O-tRNA), a nucleic acid that comprises a polynucleotide that encodes a polypeptide of interest, and an nsAA.

Examples of cell-free systems include, but are not limited to, prokaryotic lysates such as E. coli lysates, and eukaryotic lysates such as wheat germ extracts, insect cell lysates, rabbit reticulocyte lysates, rabbit oocyte lysates and human cell lysates. Eukaryotic extracts or lysates may be preferred when the resulting protein is glycosylated, phosphorylated or otherwise modified because many such modifications are only possible in eukaryotic systems. Some of these extracts and lysates are available commercially. Membranous extracts, such as the canine pancreatic extracts containing microsomal membranes, are also available which are useful for translating secretory proteins.

Reconstituted translation systems may also be used. Mixtures of purified translation factors have also been used successfully to translate mRNA into protein as well as combinations of lysates or lysates supplemented with purified translation factors such as initiation factor-1 (IF-1), IF-2, IF-3 (C or B), elongation factor T (EF-Tu), or termination factors.

Cell-free systems may also be coupled transcription/translation systems wherein DNA is introduced to the system, transcribed into mRNA and the mRNA translated as described in Current Protocols in Molecular Biology (F. M. Ausubel et al. editors, Wiley Interscience, 1993). RNA transcribed in a eukaryotic transcription system may be in the form of heteronuclear RNA (hnRNA) or 5′-end caps (7-methyl guanosine) and 3′-end poly A tailed mature mRNA, which can be an advantage in certain translation systems. For example, capped mRNAs are translated with high efficiency in the reticulocyte lysate system.

Further, a coupled transcription/translation system may be used. Coupled transcription/translation systems allow for transcription of an input DNA into a corresponding mRNA, which is in turn translated by the reaction components. For example, a system that includes a mixture containing E. coli lysate for providing translational components such as ribosomes and translation factors could be used.

Non-Standard Amino Acids (nsAAs)

Non-standard amino acids (nsAAs) are incorporated into polypeptides by the orthogonal translation systems described herein. The generic structure of an alpha amino acid is illustrated by Formula I:

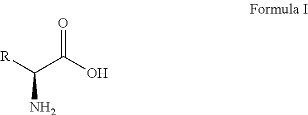

A non-standard amino acid is a non-naturally occurring amino acid, and comprises any structure having Formula I wherein the R group is any substituent other than one used in the twenty natural amino acids, which distinguish them from natural amino acids. For example, R in Formula I may comprise an alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amine, and the like, or any combination thereof.

Other non-standard amino acids include, but are not limited to, amino acids comprising a photoactivatable cross-linker, a spin-labeled amino acid, a fluorescent amino acid, a metal binding amino acid, a metal containing amino acid, a radioactive amino acid, amino acids with novel functional groups, amino acids that covalently or noncovalently interact with other molecules, photocaged amino acids, photoisomerizable amino acids, amino acids comprising biotin or a biotin analogue, glycosylated amino acids such as a sugar substituted serine, other carbohydrate modified amino acids, keto-containing amino acids, amino acids comprising polyethylene glycol or polyether, a heavy atom substituted amino acids, chemically cleavable amino acids, photocleavable amino acids, amino acids with an elongated side chains as compared to natural amino acids, including but not limited to, polyethers or long chain hydrocarbons, including but not limited to, greater than about five or greater than about ten carbons, carbon-linked sugar-containing amino acids, redox active amino acids, amino thio acid containing amino acids, and amino acids comprising one or more toxic moiety.

In certain embodiments, the non-standard amino acid is a derivative of a natural amino acid, such as tyrosine, glutamine, phenylalanine, and the like. In certain embodiments, the non-standard amino acid is a tyrosine analog. In certain embodiments, the tyrosine analogs include para-substituted tyrosines, ortho-substituted tyrosines, and meta-substituted tyrosines, wherein the substituted tyrosine comprises a keto group (including but not limited to, an acetyl group), a benzoyl group, an amino group, a hydrazine, a hydroxyamine, a thiol group, a carboxy group, an isopropyl group, a methyl group, a branched hydrocarbon, a saturated or unsaturated hydrocarbon, an O-methyl group, a polyether group, a nitro group, or the like. Multiply substituted aryl rings are also contemplated. Glutamine analogs include, but are not limited to, α-hydroxy derivatives, γ-substituted derivatives, cyclic derivatives, and amide substituted glutamine derivatives. Example phenylalanine analogs include, but are not limited to, para-substituted phenylalanines, ortho-substituted phenylalanines, and meta-substituted phenylalanines, wherein the Substituent comprises a hydroxy group, a methoxy group, a methyl group, an allyl group, an aldehyde, an azido, an iodo, a bromo, a keto group (including but not limited to, an acetyl group), or the like.

Specific examples of non-standard amino acids include, but are not limited to, a p-acetyl-L-phenylalanine (Formula II), a p-azido-L-phenylalanine (Formula III), a p-propargyl phenylalanine (Formula IV), an O-methyl-L-tyrosine, an L-3-(2-naphthyl)alanine, a 3-methylphenylalanine, an O-4-allyl-L-tyrosine, a 4-propyl-L-tyrosine, a tri-O-acetyl-GlcNAcB-serine, an L-Dopa, a fluorinated phenylalanine, an isopropyl-L-phenylalanine, a p-acyl-L-phenylalanine, a p-benzoyl-L-phenylalanine, an L-phosphoserine, a phosphono serine, a phosphonotyrosine, a p-iodo-phenylalanine, a p-bromophenylalanine, a p-amino-L-phenylalanine, and an isopropyl-L-phenylalanine.

In certain embodiments, the non-standard amino acid is an O-methyl-L-tyrosine. In certain embodiments, the non-standard amino acid is an L-3-(2-naphthyl)alanine. In certain embodiments, the non-standard amino acid is an amino-, an isopropyl-, or an O-allyl-containing phenylalanine analogue. In certain embodiments, the non-standard amino acid is acetyl-phenylalanine (AcF). In certain embodiments, the non-standard amino acid is 4-azido-phenylalanine (AzF). In certain embodiments, the non-standard amino acid is 4-propargyloxyphenylalanine (PaF). In certain embodiments, the non-standard amino acid is 4-aminophenylalanine (AmF).

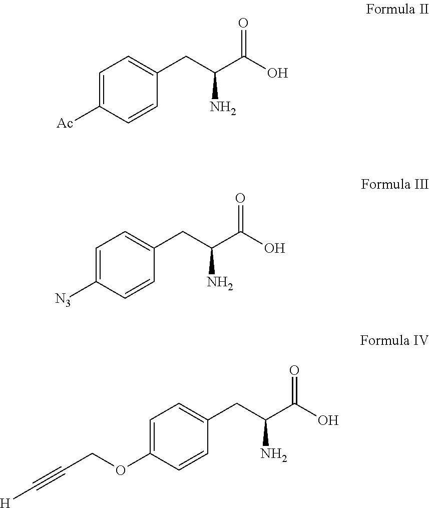 [SMILES: CC(=O)c1ccc(C[C@H](N)C(=O)O)cc1 ; [N-]=[N+]=Nc1ccc(C[C@H](N)C(=O)O)cc1 ; C#CCOc1ccc(C[C@H](N)C(=O)O)cc1]

### Nucleic Acids

The present disclosure includes a polynucleotide or set of polynucleotides comprising a nucleic acid sequence of an O-tRNA and/or encoding an O-RS described herein. The present disclosure also includes nucleic acid sequences complementary to an O-tRNA and/or an O-RS described herein.

In addition, this disclosure includes polynucleotides encoding a protein of interest described herein comprising one or more selector codon(s). In certain embodiments, the nucleic acid comprises at least one selector codon, at least two selector codons, at least three selector codons, at least four selector codons, at least five selector codons, at least six selector codons, at least seven selector codons, at least eight selector codons, at least nine selector codons, or even ten or more selector codons.

In an aspect, described herein is a polynucleotide comprising a nucleic acid sequence at least 85% identical to the sequence set forth in SEQ ID NO: 1 and comprising a deletion of the cytosine located at nucleic acid position 16 of the O-tRNA, wherein the nucleic acid positions correspond to the sequence set forth in SEQ ID NO: 1. In certain embodiments, the nucleic acid sequence is at least 90% identical, at least 91% identical, at least 92% identical, at least 93% identical, at least 94% identical, at least 95% identical, at least 96% identical, at least 97% identical, at least 98% identical, or at least 99% identical to the sequence set forth in SEQ ID NO: 1. In an embodiment, the nucleic acid sequence comprises an adenine at nucleic acid position 52 and a thymine or uracil at nucleic acid position 62, wherein the nucleic acid positions correspond to the sequence set forth in SEQ ID NO: 1. In an embodiment, the polynucleotide comprises the nucleic acid sequence consisting of the sequence set forth in SEQ ID NO: 2. In an embodiment, the nucleic acid sequences comprises a cytosine at amino acid positions 3 and 6; a thymine or uracil at nucleic acid position 7, an adenosine at nucleic acid position 66, and a guanine at nucleic acid positions 67 and 70, wherein the nucleic acid positions correspond to the sequence set forth in SEQ ID NO: 1. In an embodiment, the polynucleotide comprises a nucleic acid sequence consisting of the sequence set forth in SEQ ID NO: 3. In an embodiment, the polynucleotide comprises a nucleic acid sequence consisting of the sequence set forth in SEQ ID NO: 4. In an embodiment, the polynucleotide comprises the nucleic acid sequence CAGAGGGCAG at nucleic acid positions 13 to 22, wherein the nucleic acid positions correspond to the sequence set forth in SEQ ID NO: 1. In an embodiment, the polynucleotide comprises a nucleic acid sequence consisting of a sequence set forth in any one of SEQ ID NOs: 2-16.

In an aspect, described herein is a polynucleotide comprising a nucleic acid sequence of an O-tRNA comprising a nucleic acid sequence set forth in any one of SEQ ID NOs: 2-31. In an embodiment, the polynucleotides further comprise a nucleic acid sequence complementary to an O-tRNA comprising a nucleic acid sequence set forth in any one of SEQ ID NOs: 2-31.

In an aspect, described herein are polynucleotides comprising a nucleic acid sequence encoding an O-RS comprising an amino acid sequence set forth in SEQ ID NO: 35 or 39. In certain embodiments, the polynucleotides further comprise a nucleic acid sequence complementary to a nucleic acid sequence encoding an O-RS comprising an amino acid sequence set forth in SEQ ID NO: 35 or 39.

In an aspect, described herein is a polynucleotide or set of polynucleotides comprising a nucleic acid sequence of an O-tRNA comprising a nucleic acid sequence set forth in any one of SEQ ID NOs: 2-31 and a nucleic acid sequence encoding the O-RS comprising an amino acid sequence set forth in SEQ ID NO: 35 or 39.

### Vectors

In certain embodiments, a vector (e.g., a plasmid, a cosmid, a phage, a virus, etc.) comprises a polynucleotide described herein. In certain embodiments, the vector is an expression vector. In certain embodiments, the expression vector includes a promoter operably linked to one or more of the polynucleotides described herein. In certain embodiments, a cell comprises a vector that includes a polynucleotide disclosed herein.

### Cells

In certain embodiments, this disclosure includes cells comprising an O-tRNA, an O-RS, an nsAA, and/or an OTS described herein. The cells described herein include any of, e.g., prokaryotic cells (e.g., E. coli.), non-prokaryotic cells, mammalian cells, yeast cells, fungus cells, plant cells, insect cells, etc. In certain embodiments, the cell encodes a mutation in an EF-Tu. In certain embodiments, the cell has reduced expression of Release Factor 1 compared to an otherwise identical wild-type cell.

In certain embodiments, the cells containing the O-tRNA, O-RS, nsAA, and/or OTS described herein are cells as described in U.S. Pat. Nos. 9,617,335, 10,465,197, 10,604,761, and U.S. application Ser. No. 15/261,984, U.S. application Ser. No. 16/871,736, which are incorporated herein by reference in their entireties.

In certain aspects, described herein are cells comprising a nucleic acid that comprises a polynucleotide that encodes a polypeptide of interest, where the polynucleotide comprises a selector codon that is recognized by the O-tRNA. In one aspect, the yield of the polypeptide of interest comprising the nsAA is, e.g., at least 2.5%, at least 5%, at least 10%, at least 25%, at least 30%, at least 40%, 50% or more, of that obtained for the naturally occurring polypeptide of interest from a cell in which the polynucleotide lacks the selector codon. In another aspect, the cell produces the polypeptide of interest in the absence of the nsAA, with a yield that is, e.g., less than 50%, less than 35%, less than 30%, less than 20%, less than 15%, less than 10%, less than 5%, less than 2.5%, etc., of the yield of the polypeptide in the presence of the nsAA.

Compositions that include a cell comprising an orthogonal tRNA (O-tRNA) are also a feature of the invention. Typically, the O-tRNA mediates incorporation of an nsAA into a protein that is encoded by a polynucleotide that comprises a selection codon that is recognized by the O-tRNA in vivo. In one embodiment, the O-tRNA mediates the incorporation of the nsAA into the protein with, e.g., at least 45%, at least 50%, at least 60%, at least 75%, at least 80%, at least 90%, at least 95%, or even 99% or more the efficiency of a tRNA that comprises or is processed in a cell from a polynucleotide sequence as set forth in SEQ ID NO: 65. In another embodiment, the O-tRNA comprises or is processed from a polynucleotide sequence as set forth in SEQ ID NO: 65, or a conservative variation thereof. In yet another embodiment, the O-tRNA comprises a recyclable O-tRNA.

In certain embodiments, the cells described herein have the ability to synthesize proteins that comprise nsAAs in large useful quantities. For example, proteins comprising an nsAA can be produced at a concentration of, e.g., at least 10 μg/liter, at least 50 μg/liter, at least 75 μg/liter, at least 100 μg/liter, at least 200 μg/liter, at least 250 μg/liter, or at least 500 g/liter or more of protein in a cell extract, a buffer, a pharmaceutically acceptable excipient, and/or the like. In certain embodiments, a composition of the invention includes, e.g., at least 10 kg, at least 50 g, at least 75 g, at least 100 g, at least 200 kg, at least 250 g, or at least 500 kg or more of protein that comprises an nsAA.

Once a recombinant host cell strain has been established (i.e., one or more expression vectors comprising polynucleotide sequences for the O-tRNA and/or O-RT has been introduced into the host cell and host cells with the proper expression construct are isolated), the recombinant host cell strain is cultured under conditions appropriate for production of the polypeptide of interest. As will be apparent to one of skill in the art, the method of culture of the recombinant host cell strain will be dependent on the nature of the expression construct utilized and the identity of the host cell. Recombinant host cells may be cultured in batch or continuous formats, with either cell harvesting (in the case where the polypeptide of interest accumulates intracellularly) or harvesting of culture supernatant in either batch or continuous formats. For production in prokaryotic host cells, batch culture and cell harvest can be performed. In certain embodiments, fed batch fermentation culturing conditions are used.

Polypeptides Comprising at Least One nsAA

Proteins (or polypeptides of interest) with at least one nsAA are also disclosed herein. In certain embodiments, a protein with at least one nsAA includes at least one post-translational modification. In an embodiment, the at least one post-translational modification comprises attachment of a molecule (e.g., a dye, a polymer, e.g., a derivative of polyethylene glycol, a photocrosslinker, a cytotoxic compound, an affinity label, a derivative of biotin, a resin, a second protein or polypeptide, a metal chelator, a cofactor, a fatty acid, a carbohydrate, a polynucleotide (e.g., DNA, RNA, etc.), etc.) comprising a second reactive group by a [3+2] cycloaddition to the at least one nsAA comprising a first reactive group. For example, the first reactive group is an alkynyl moiety (e.g., in the nsAA p-propargyloxyphenylalanine) (this group is also sometimes refer to as an acetylene moiety) and the second reactive group is an azido moiety. In another example, the first reactive group is the azido moiety (e.g., in the nsAA p-azido-L-phenylalanine) and the second reactive group is the alkynyl moiety. In certain embodiments, a protein of the invention includes at least one nsAA (e.g., a keto nsAA) comprising at least one post-translational modification, where the at least one post-translational modification comprises a saccharide moiety. In certain embodiments, the post-translational modification is made in vivo in a cell. Thus, in certain embodiments, the protein(s) comprising non-standard amino acids that are produced are processed and modified in a cell-dependent manner. This provides for the production of proteins that are stably folded, glycosylated, or otherwise modified by the cell.

In certain embodiments, the protein includes at least one post-translational modification that is made in vivo by a cell, where the post-translational modification is not made by a prokaryotic cell. Examples of post-translational modifications include, but are not limited to, acetylation, acylation, lipid-modification, palmitoylation, palmitate addition, phosphorylation, glycolipid-linkage modification, and the like. In an embodiment, the post-translational modification comprises attachment of an oligosaccharide to an asparagine by a GlcNAc-asparagine linkage (e.g., where the oligosaccharide comprises (GlcNAc-Man)₂-Man-GlcNAc-GlcNAc, and the like). In another embodiment, the post-translational modification comprises attachment of an oligosaccharide (e.g., Gal-GalNAc, Gal-GlcNAc, etc.) to a serine or threonine by a GalNAc-serine, a GalNAc-threonine, a GlcNAc-serine, or a GlcNAc-threonine linkage. In certain embodiments, a protein or polypeptide of the invention can comprise a secretion or localization sequence, an epitope tag, a FLAG tag, a polyhistidine tag, a GST fusion, and/or the like.

Typically, the proteins are, e.g., at least 60%, at least 70%, at least 75%, at least 80%, at least 90%, at least 95%, or even at least 99% or more identical to any available protein (e.g., a therapeutic protein, a diagnostic protein, an industrial enzyme, or portion thereof, and/or the like), and they comprise one or more nsAA. In an embodiment, disclosed herein are composition comprising a protein or polypeptide of interest comprising at least one nsAA and an excipient (e.g., a buffer, a pharmaceutically acceptable excipient, etc.).

Examples of a protein (or polypeptides) of interest include, but are not limited to, e.g., an antibody or antigen binding fragment thereof, a cytokine, a growth factor, a growth factor receptor, an interferon, an interleukin, an inflammatory molecule, an oncogene product, a peptide hormone, a signal transduction molecule, a steroid hormone receptor, a transcriptional modulator protein (e.g., a transcriptional activator protein (such as GAL4), or a transcriptional repressor protein, etc.) or a portion thereof. In certain embodiments, the protein of interest comprises a therapeutic protein, a diagnostic protein, an industrial enzyme, or a portion(s) thereof.

In an embodiment, the protein of interest that is produced by the methods described herein are further modified through the one or more nsAA(s). For example, the nsAA can be modified through, e.g., a nucleophilic-electrophilic reaction, through a [3+2]cycloaddition, etc. In certain embodiments, the protein produced by the methods described herein is modified by at least one post-translational modification (e.g., N-glycosylation, 0-glycosylation, acetylation, acylation, lipid-modification, palmitoylation, palmitate addition, phosphorylation, glycolipid-linkage modification, and the like) in vivo.

In an aspect, the protein or polypeptide of interest (or portion thereof) is encoded by a nucleic acid. Typically, the nucleic acid comprises at least one selector codon, at least two selector codons, at least three selector codons, at least four selector codons, at least five selector codons, at least six selector codons, at least seven selector codons, at least eight selector codons, at least nine selector codons, or even ten or more selector codons.

### Kits

Kits are also a feature of this disclosure. For example, a kit for producing a protein that comprises at least one nsAA is provided. In certain embodiments, the kit includes an O-tRNA or a polynucleotide sequence coding for an O-tRNA or comprising a O-tRNA, and/or an O-RS or a polynucleotide sequence encoding an O-RS. In certain embodiments, the kit includes cells comprising a polynucleotide sequence comprising an O-tRNA, and/or an O-RS or a polynucleotide sequence encoding an O-RS. In certain embodiments, the cells comprise a polynucleotide encoding a polypeptide or protein of interest. In certain embodiments, the kit further includes at least one nsAA. In certain embodiments, the kit further comprises instructional materials for producing the protein.

Methods for Incorporating nsAA into Polypeptides at Specific Locations

Included herein are methods for producing a polypeptide comprising at least one nsAA, comprising expressing in a cell an O-tRNA comprising a nucleic acid sequence at least 85% identical to the sequence set forth in SEQ ID NO: 1 and comprising a deletion of the cytosine located at nucleic acid position 16 of the O-tRNA, wherein the nucleic acid positions correspond to the sequence set forth in SEQ ID NO: 1; and wherein the O-tRNA is capable of being aminoacylated with at least one non-standard amino acid (nsAA) by an O-RS.

In certain aspects, described herein are methods for producing a polypeptide comprising at least one nsAA, comprising providing: i) an O-tRNA comprising a nucleic acid sequence at least 85% identical to the sequence set forth in SEQ ID NO: 1 and comprising a deletion of the cytosine located at nucleic acid position 16 of the O-tRNA; wherein the nucleic acid positions correspond to the sequence set forth in SEQ ID NO: 1; and wherein the O-tRNA is capable of being aminoacylated with at least one non-standard amino acid (nsAA) by an O-RS; ii) an O-RS; wherein the O-RS aminoacylates the O-tRNA with the nsAA; and iii) a polynucleotide encoding the polypeptide, wherein the polynucleotide comprises at least one selector codon; and wherein the O-tRNA recognizes the selector codon.

In certain embodiments, the nucleic acid sequence is at least 90% identical, at least 91% identical, at least 92% identical, at least 93% identical, at least 94% identical, at least 95% identical, at least 96% identical, at least 97% identical, at least 98% identical, or at least 98.7% identical to the sequence set forth in SEQ ID NO: 1.

In certain embodiments, the O-tRNA comprises an adenine at nucleic acid position 53 and a uracil at nucleic acid position 63, wherein the nucleic acid positions correspond to the sequence set forth in SEQ ID NO: 1. In certain embodiments, the O-tRNA comprises a nucleic acid sequence consisting of the sequence set forth in SEQ ID NO: 2. In certain embodiments, the O-tRNA comprises a cytosine at nucleic acid positions 3 and 6; a uracil at nucleic acid position 7, an adenosine at nucleic acid position 67, and a guanine at nucleic acid positions 68 and 71, wherein the nucleic acid positions correspond to the sequence set forth in SEQ ID NO: 1. In certain embodiments, the O-tRNA comprises a nucleic acid sequence consisting of the sequence set forth in SEQ ID NO: 3. In certain embodiments, the O-tRNA comprises a nucleic acid sequence consisting of the sequence set forth in SEQ ID NO: 4. In certain embodiments, the O-tRNA comprises the sequence CAG-AGGGCAG at nucleic acid positions 13 to 23, wherein the nucleic acid positions correspond to the sequence set forth in SEQ ID NO: 1. In certain embodiments, the O-tRNA comprises a nucleic acid sequence set forth in any one of SEQ ID NOs: 2-16.

In certain embodiments, the method further comprises expressing an O-RS described herein in the cell.

In certain embodiments, the protein or polypeptide of interest (or portion thereof) is comprises at least one nsAA, at least two nsAAs, at least three nsAAs, at least four nsAAs, at least five nsAAs, at least six nsAAs, at least seven nsAAs, at least eight nsAAs, at least nine nsAAs, or even ten or more nsAAs.

This disclosure also provides methods for producing, in a cell, at least one protein of interest comprising at least one nsAA. The methods include, e.g., growing, in an appropriate medium, a cell that comprises a nucleic acid that comprises at least one selector codon and encodes the protein.

In an embodiment, the method further includes incorporating into the protein of interest the nsAA, where the nsAA comprises a first reactive group; and contacting the protein with a molecule (e.g., a dye, a polymer, e.g., a derivative of polyethylene glycol, a photocrosslinker, a cytotoxic compound, an affinity label, a derivative of biotin, a resin, a second protein or polypeptide, a metal chelator, a cofactor, a fatty acid, a carbohydrate, a polynucleotide (e.g., DNA, RNA, etc.), etc.) that comprises a second reactive group. The first reactive group reacts with the second reactive group to attach the molecule to the nsAA through a [3+2] cycloaddition. In an embodiment, the first reactive group is an alkynyl or azido moiety and the second reactive group is an azido or alkynyl moiety. For example, the first reactive group is the alkynyl moiety (e.g., in nsAA p-propargyloxyphenylalanine) and the second reactive group is the azido moiety. In another example, the first reactive group is the azido moiety (e.g., in the nsAA p-azido-L-phenylalanine) and the second reactive group is the alkynyl moiety.

The present invention also provides methods for producing at least one protein in a prokaryotic (e.g., Eubacteria) or eukaryotic (e.g., yeast, protist mammalian, plant, or insect) translation system. In certain embodiments, a cell, e.g., an E. coli cell, comprising the tRNA of the present invention includes such a translation system. The translation system is provided with the at least one non-standard amino acid, thereby producing at least one protein containing at least one non-standard amino acid. The compositions and methods described here can be used with non-standard amino acids, e.g., providing specific spectroscopic, chemical, or structural properties to proteins using any of a wide array of side chains. These compositions and methods are useful for the site-specific incorporation of non-standard amino acids via selector codons, e.g., stop codons, four base codons, and the like. The translation system is also provided with an orthogonal tRNA (O-tRNA), that functions in the translation system and recognizes the at least one selector codon and an orthogonal aminoacyl tRNA synthetase (0-RS), that preferentially aminoacylates the O-tRNA with the at least one non-standard amino acid in the translation system.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B(1992).

### Example 1: Selection Scheme for the Directed Evolution of O-RS

In this Example, incorporation of nsAAs into proteins utilizes three components: 1) a suitable nsAA, 2) a tRNA molecule that is orthogonal to native tRNA molecules (i.e., the tRNA is not readily aminoacylated by native tRNA synthetases in the cell) which has an engineered anticodon to recognize the amber codon, and 3) an orthogonal aminoacyl tRNA synthetase that can aminoacylate the nsAA onto the orthogonal tRNA (and that will not readily aminoacylate the nsAA onto native tRNAs). The following sections describe the identification of a suitable system for robust nsAA incorporation at amber codons (TAG) in E. coli SoluPro®.

Candidate amino acids and orthogonal tRNA/aminoacyl tRNA synthetase pairs were selected to serve as a starting point for additional engineering:

### Choice of Amino Acid

Two nsAAs were selected as candidates: para-Acetyl-L-phenylalanine (pAcF) and para-Azido-L-phenylalanine (pAzF). These amino acids were attractive as they are 1) commercially available, 2) relatively non-toxic to E. coli, 3) compatible with the M. jannaschii tRNA-synthetase/tRNA pair (described below), and 4) once incorporated into proteins, can facilitate chemoselective ligation reactions to attach novel moieties to the protein under relatively mild conditions. pAcF was further investigated due to its relatively lower cost.

Orthogonal tRNA/Aminoacyl tRNA Synthetase Pair

The M. jannaschii tyrosyl-tRNA synthetase (MjtRNA^{Tyr}_{CUA}/MjYRS) pair has been the most extensively used starting point for the evolution of Orthogonal Translation Systems (OTSs) that incorporate non-standard amino acids in E. coli. The MjYRS does not aminoacylate any endogenous E. coli tRNAs with tyrosine, but aminoacylates a mutant tyrosine amber suppressor (mutRNA_{CUA}). By applying a combination of synergized approaches, such as high-throughput cloning, FACS sorting, NGS analysis, in vitro evolution, synthetic biology, structural biology, and artificial intelligence, a series of variants for MjtRNA^{Tyr}_{CUA}/MjYRS pairs was identified with significantly enhanced activity for the incorporation of non-standard amino acids into proteins. Candidate MjtRNA^{Tyr}_{CUA}/MjYRS pairs were evaluated for incorporation efficiency in Example 2.

Mj tyrosyl-tRNA synthetase interacts with the anticodon present on the corresponding Mj tyrosyl-tRNA. Because the anticodon is mutated in these systems to direct the tRNA toward the amber codon (TAG) instead of the native tyrosine codon (TAC), the modification impacts the aminoacyl tRNA synthetase/tRNA interaction and decreases the efficiency of tRNA charging. Crystallographic and associated biochemical studies of the M. jannaschii aminoacyl tRNA synthetase/tRNA complex have identified mutation (D286R) that can largely restore the interaction between the tRNA-synthetase and mutant tRNA. It has been discovered that a new E9 synthetase, comprising a D286R mutation and an I15V mutation, results in an efficient tRNA-synthetase for nsAA incorporation at the amber codon.

Evolution of Optimal tRNA

Starting with the M. jannaschii tyrosyl-tRNA, several specific modifications were introduced, including swapping out the natural CUA anticodon (to enable the tRNA to pair with the TAG codon), and five mutations that were previously identified in an in vivo screen to bestow greater orthogonality in E. coli upon the Mj tyrosyl-tRNA. A challenge to using the Mj tyrosyl-tRNA in E. coli is that it should interact efficiently with E. coli's native elongation factor Tu (EF-Tu) in order to be transported to the ribosome and utilized in protein synthesis. Because significant sequence differences exist between E. coli tRNAs and archaeal tRNAs, the interaction between Mj tyrosyl-tRNA and E. coli EF-Tu is not very efficient. In order to improve this efficiency, a library of tRNA sequences was generated (see “library_tRNA” SEQ ID NO: 34) where diversity was incorporated into nucleotides that are known to interact with EF-Tu4.

This tRNA library was initially screened in a two-fold selection process. The tRNA library was introduced along with the Mj E9_I15V_D286R aminoacyl tRNA synthetase on a plasmid containing a fusion of the chloramphenicol acyltransferase (CAT) gene and the uracil phosphoribosyltransferase (UPRT) gene. The fusion protein contains multiple TAG codons throughout the sequence. This fusion protein serves as a positive/negative selection cassette. CAT is a chloramphenicol resistance protein, conferring survival in the presence of chloramphenicol, and UPRT converts 5-fluorouracil (5-FU) to a toxic compound that kills the cell. Cells were cultivated in the absence of the amino acid pAcF and the presence of 5-FU. Any tRNAs in the library that can be charged with any amino acid by any aminoacyl tRNA synthetase (i.e., are not truly orthogonal) led to amber suppression, expression of UPRT, and subsequent death of the cell. In this manner, non-orthogonal tRNAs were rapidly eliminated from the library. Surviving cells were cultivated in the presence of the amino acid pAcF and chloramphenicol. In this case, tRNAs that can be charged with pAcF by Mj E9_I15V_D286R conferred chloramphenicol resistance to the cell and survived. The enriched tRNA library was subcloned into a new plasmid containing Mj E9_I15V_D286R aminoacyl tRNA synthetase and a GFP gene containing internal TAG codons under control of the arabinose paraBAD promoter. Incorporation of pAcF at the TAG codons upon induction led to fluorescent protein production. The cells were sorted to collect the brightest members of the population, which corresponded to the most efficient nsAA incorporators.

For additional validation, discrete tRNA designs from the sorting were used to incorporate pAcF into a model molecule (such as Herceptin or TRAST-Fab). Successful incorporation were monitored by SDS-PAGE and confirmed by peptide mapping. These results show that the tRNA designs described herein can efficiently and with high fidelity incorporate nsAAs into proteins of interest.

### Example 2: nsAA Incorporation at Various Strategic Sites in Trastuzumab Under Shake-Flask or Fed-Batch Fermentation Conditions Using Candidate OTSs

To examine the utility of the engineered OTSs in the nsAA incorporation for biologics, the constructs harboring the candidate variants of MjtRNA^{Tyr}_{CUA}/MjYRS pairs were co-transformed with a Trastuzumab expressing construct. The constructs containing candidate OTSs contain a low-copy number origin of replication pACYC and a tetracycline resistance cassette. O-tRNA expression is driven by the constitutive promoter plpp and terminated by rrnB1 terminator while the expression of O-RS is controlled by the constitutive promoter pgln and terminated by rrnC1 terminator.

Cells were cultured in LB (10 g/L tryptone, 5 g/L yeast extract, 10 g/L NaCl). Tetracycline (15 μg/mL), kanamycin (50 μg/mL), arabinose (250 μM), propionate (20 mM) were added as appropriate. Amino acids 4-acetyl-L-phenylalanine (A206865), 4-propargyloxy-L-phenylalanine (A721556), 4-Amino-L-phenylalanine hydrate (A943099) were purchased from Ambeed. 4-azido-L-phenylalanine (909564) was purchased from Sigma-Aldrich. Stock solutions of L-tyrosine was prepared at 500 mM in dH2O. For 4-acetyl-L-phenylalanine, 4-propargyloxy-L-phenylalanine, 4-Amino-L-phenylalanine hydrate and 4-azido-L-phenylalanine, a 500 mM stock solution was prepared in in 1 M NaOH solution. All amino acid stocks were sterilized using 0.22 μm filters. All stock solutions of nsAA were prepared as 500×.

To inoculate the positive/negative strains in liquid culture, in the biosafety cabinet, 5 mL LB broth was added to each tube using a serological pipet, 5 uL of appropriate antibiotic to each tube as added, a P1000 tip was used to stab the frozen glycerol stock. The tip was placed in a 5 mL TB+KAN tube and mixed well 3 times. Culture tubes were placed in a 30° C. shaking incubator at 270 rpm for 20-24 hours. 3 mL of complete fermentation production media was added per well of a 24-DW plate(s) using a serological pipet. The complete fermentation media containing 0.5 mM nsAA. The fermentation media was inoculated with 200 μL of the cultures with an optical density of 3 and mixed 5 times. Plates were covered with an aero-seal and cultures incubated at 27° C. with shaking at 270 rpm for 24 h.

Optical density of the cultures was measured at the time of harvest. After incubation, samples were removed from the incubator and placed on ice. A 50× dilution in an OD plate was prepared. Wells were filled with 196 μL of dH2O, 1 row for each row in the induction plate. A P-20 or P-10 multi-channel pipette was used to add two aliquots of 4 uL for every induction well to the OD plate. The OD plate was measured on the SpectraMax plate reader using the OD600 protocol with both pathlength correction and blank subtraction turned on.

At the time of harvest, 1100 uL 60% glycerol and 2200 uL culture was added to a new well of a 24 DW plate for a final concentration of 20% glycerol, and mixed. 500 uL of the glycerol-sample mixture was added to 0.7 mL matrix tubes in a tube rack. Harvested glycerol stocks were stored at −80° C.

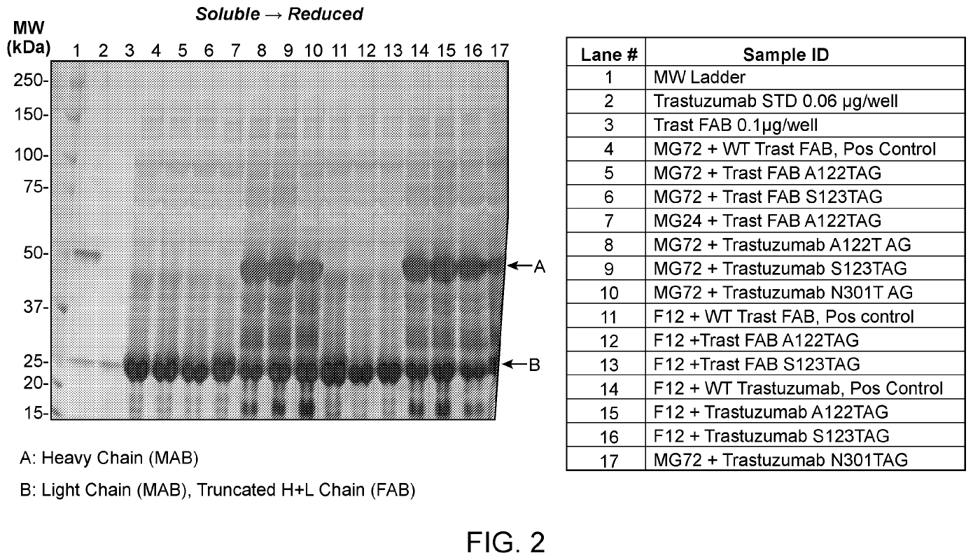

Cells were pelleted for downstream analysis. Leftover culture of each library was used to harvest in the tube for subsequent analysis (for ‘Pre-sort’ sample). The cell pellet samples were further lysed and analyzed by western blot. More specifically, the samples were normalized to OD10 by varying the amount of lysis buffer (50 mM Tris, 200 mM NaCl, pH 7.4, 1% w/v Octylglucoside, 0.08 μL/mL rLysozyme, 0.08 μL/mL Benzonase Nuclease) added to each sample. The samples were lysed for one hour. The lysates were centrifuged at 3300 g at 4° C. for 30 minutes, and the supernatants (soluble fraction) were collected. The soluble fractions were treated with non-reducing reagent (10 mM IAA in 1×LDS NuPAGE Sample Buffer) and reducing reagent (50 mM DTT in 1×LDS NuPAGE Sample Buffer). The trastuzumab and trastuzumab FAb standards (Trast Fab standards) were treated with both non-reducing (10 mM IAA in 1×LDS NuPAGE Sample Buffer) and reducing (50 mM DTT in 1×LDS NuPAGE Sample Buffer) reagents. SDS-PAGE was performed after ProA/PhyTip purification of trastuzumab with nsAA incorporation at various strategic sites produced under shake-flask fermentation conditions (FIG. 2).

Fermentation scale-up was performed to demonstrate successful incorporation of the nonstandard amino acid, para-acetylphenylalanine into the strategic site HC_A122 on trastuzumab using an OTS under the standard Dasbox bioreactor fermentation conditions. The strain BR7 harboring a candidate amber codon suppression system was tested to see if it could produce a comparable titer of nsAA-incorporated trastuzumab compared to positive control strain BR2, which encodes the previously validated best genotype for the production of wild type trastuzumab.

The following protocol was used for scale-up fermentation. Control settings, parameter setpoints, and the bioreactor scripts were verified. The pH control was started or the set point raised to the correct value. All Control processes were verified to be turned on and operating properly, including: the DO Control, pH Control, Agitation, Gassing, Temperature Control, Pump A-C. All current process parameter setpoints were verified. Seed flasks and supplies were transferred into the sterile laminar flow hood. For every bioreactor inoculated, 1×20-gauge needle and 1×10 mL or 5 mL syringe was used.

For every seed culture flask prepared, 1×50 mL serological pipette, 1×5 mL serological pipette, 1×50 mL conical tube and 1×1.7 mL microcentrifuge tube was used. The OD600 of seed culture was measured and the inoculum required to reach an OD of 0.1 in the reactor was then calculated. 1 mL of culture was transferred from the flask into a microcentrifuge tube. Seed flask ODs and inoculation volume were recorded. Seed culture was aliquoted into labeled 50 mL conical vials using a 50 mL serological pipette

Equipment and bioreactors were prepared for inoculation using the following protocol. Two luer caps from addition ports on either side of pH probe were removed. The inoculation culture was gently inverted in the syringe before addition to the reactors. The inoculate culture was expelled into each bioreactor one at a time through either a triport tube or a rubber septum. The inoculation clock on DASware control was started as soon as the inoculation of bioreactors is completed.

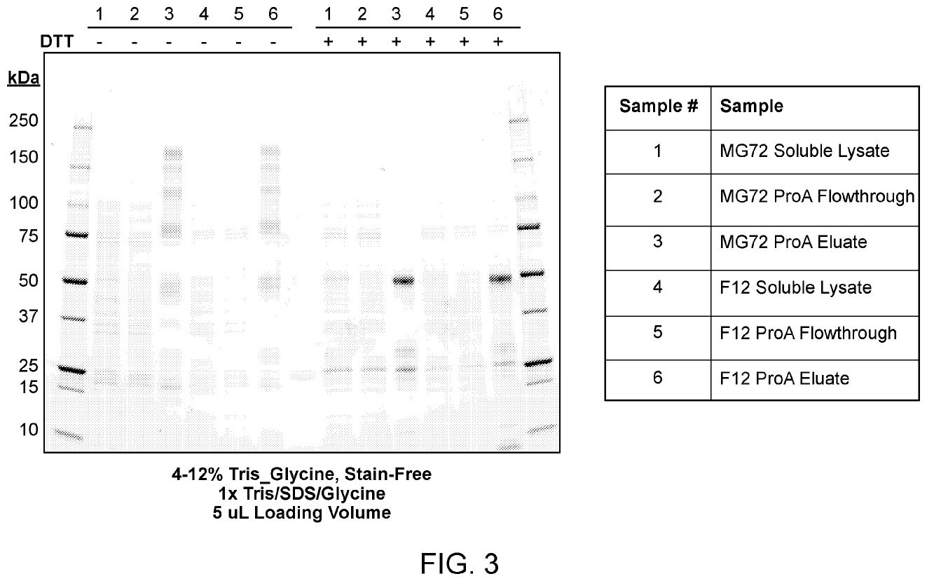

At the time of harvest, cells were pelleted for downstream analysis. Culture of each bioreactor was used to harvest in the tube for subsequent analysis (for ‘Pre-sort’ sample). Cell were centrifuged at 3300×g, 4° C. for 7 min. These cell pellet samples were further lysed and purified using commercially available PhyTip protocol and analyzed by western blot. FIG. 3 shows the results by SDS-PAGE after AKTA purification of trastuzumab with substitution of S123 for p-acetylphenylalanine produced under shake-flask fermentation conditions.

These results show cells harboring an amber codon orthogonal translation system described herein can efficiently produce an antibody harboring p-acetylphenylalanine under shake-flask fermentation conditions.

### Example 3: Evaluation of the Incorporation Efficiency of Candidate OTSs

The performance of orthogonal translation systems (OTSs) varies greatly in terms of the efficiency and accuracy of nsAA incorporation. To enable rapid and systematic comparisons of these critical parameters, a toolkit for characterizing any Escherichia coli OTS that reassigns the amber stop codon (TAG) was used. It assesses OTS performance by measuring the efficiency, i.e., relative readthrough efficiency (RRE) and fidelity, i.e., maximum misincorporation frequency (MMF), in the presence and absence of the nsAA of interest. The relative readthrough efficiency (RRE) of the TAG codon is the GFP/RFP fluorescence ratio for the mcherryTAG assay plasmid divided by the GFP/RFP fluorescence ratio for the mcherryTAC control plasmid. For an efficient aaRS-tRNA pair, the RRE when the nsAA is present in the media should approach or surpass a value of one, as this metric reflects how well the TAG amber codon is translated compared to the TAC tyrosine codon. The fidelity of an OTS was evaluated by comparing the RRE values obtained with and without nsAA present. Specifically, an (in)fidelity metric, the maximum misincorporation frequency (MMF), is calculated by dividing the RRE when nsAA is not added to the growth media by the RRE when nsAA is present. An ideal OTS has an MMF value of zero, reflecting that no GFP is produced unless the nsAA is present. Note, however, that MMF was a very strict measure of fidelity. Some engineered aaRS tRNA pairs are known to incorporate mostly the nsAA when it is provided at a sufficiently high concentration, but to nonspecifically aminoacylate the tRNA with a standard amino acid instead when it is more abundant.

Each plasmid encoded mRFP1 and sfGFP fused into a single reading frame via a flexible peptide linker. In the control plasmid, mcherryTAC, the DNA sequence encoding the linker contained a TAC tyrosine codon. The assay plasmid mcherryTAG was identical except for a single point mutation that converted the TAC into a TAG amber codon to direct incorporation of the nsAA by the OTS. In this configuration, GFP signaled readthrough of the focal TAC/TAG codon, and RFP served as an internal control for any variance in overall protein production.

The mcherryTAC (control) and mcherryTAG (assay) plasmids were created from the pBR backbone. This vector contains a kanamycin resistance cassette, the araC repressor gene, and a pBaD promoter. Plasmid construction began with pSOL_GFP, in which the wild type GFP cloned into pSOL and controlled by a pBAD promoter. The coding sequence for mCherry was amplified using PCR primers in which the linker sequence containing the TAC or TAG test codon were also appended and introduced. Gibson assembly was used to assemble the mCherry inserts and pSOL_GFP backbone. Assembly reactions were transformed into EPI300 E. coli (Lucigen), and the kit plasmids were NGS sequenced for verification.

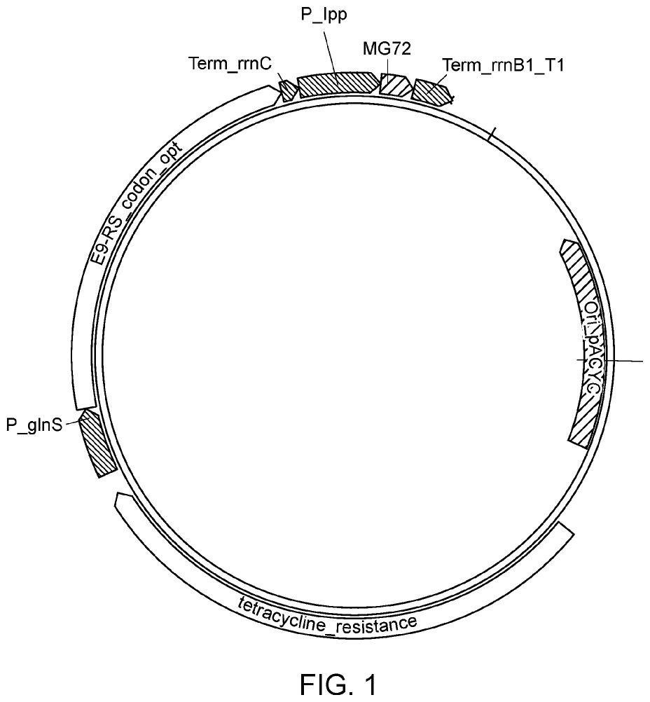

As shown in FIG. 1, the constructs containing candidate OTSs contained a low-copy number origin of replication pACYC and a tetracycline resistance cassette. Expression of O-tRNA was driven by the constitutive promoter plpp and terminated by rrnB1 terminator while the expression of O-RS was controlled by the constitutive promoter pgln and terminated by rrnC1 terminator. The N149 codon of wtGFP was mutated to an amber codon.

Cells were cultured in LB (10 g/L tryptone, 5 g/L yeast extract, 10 g/L NaCl). Tetracycline (15 μg/mL), kanamycin (50 μg/mL), arabinose (250 μM), propionate (20 mM) were added as appropriate. Amino acids 4-acetyl-L-phenylalanine (A206865), 4-propargyloxy-L-phenylalanine (A721556), 4-Amino-L-phenylalanine hydrate (A943099) were purchased from Ambeed. 4-azido-L-phenylalanine (909564) was purchased from Sigma-Aldrich. Stock solutions of L-tyrosine was prepared at 500 mM in dH2O. For 4-acetyl-L-phenylalanine, 4-propargyloxy-L-phenylalanine, 4-Amino-L-phenylalanine hydrate and 4-azido-L-phenylalanine, a 500 mM stock was prepared in a 1 M NaOH solution. All amino acid stocks were sterilized using 0.22 μm filters. All stock solutions of nsAA were prepared as 500×.

Each OTS system was constructed by using Golden Gate assembly (New England Biolabs) to clone the respective O-tRNA mutants into plasmids. Each OTS plasmid was transformed separately into E. coli strain EB114, which already contained either mcherryTAC (control) and mcherryTAG (assay) plasmid and that were made electrocompetent by 10% glycerol washes. The aaRS and tRNA genes in these clones were sequenced using Illumina technology to verify that no mutations had occurred in the OTS cassette prior to testing them using the nsAA incorporation measurement kit.

For kit assays, strains were revived from −80° C. glycerol stocks in 10 mL LB in 50 mL Erlenmeyer flasks with kanamycin and tetracycline. These cultures were incubated at 37° C. with orbital shaking over a 1-inch diameter at RPM for 24 h. From these preconditioned cultures, a diluted culture was prepared by concentrating cells in 500 μL of culture by centrifugation at 4° C., decanting the supernatant, and then adding 10 mL of fresh media with antibiotics, arabinose and +/−nsAA. This procedure creates an overall 1:100 dilution in fresh media. Cultures lacking nsAA were supplemented with an equivalent amount of sterile dH2O water to achieve a consistent LB concentration. Sample blanks+/−nsAA were prepared in an identical way, but omitting cells.

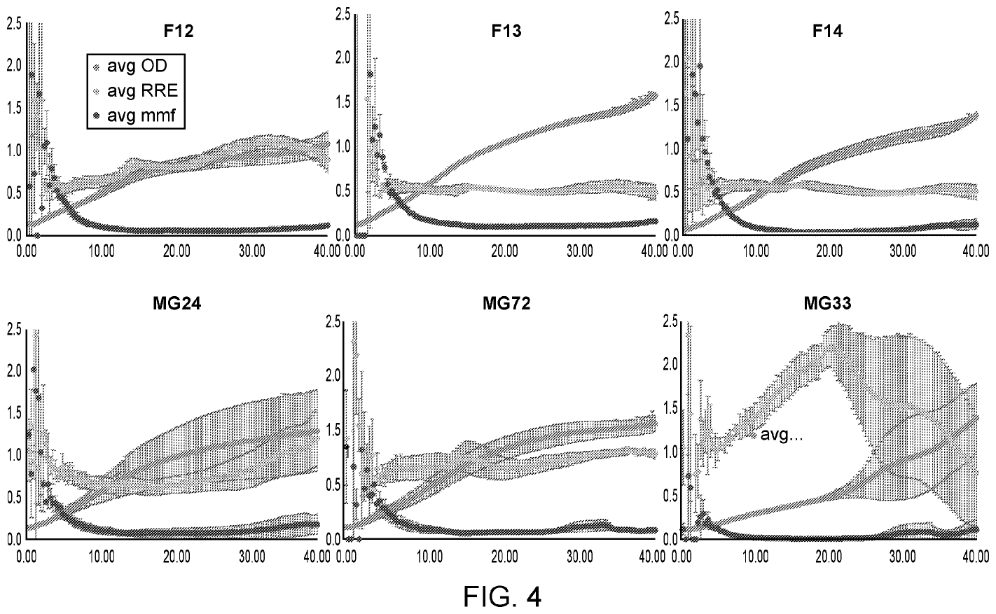

Fluorescence and OD readings were made in using an Infinite Enspire microplate reader (PerkinElmer). To test the candidate OTSs, four biological replicates were compared. For each experiment, a Costar #3631 black clear-bottom 96-well plate was filled with 150 μL aliquots of each sample and blank to be tested. The assay was run for 30 h in the microplate reader while it was incubated continuously at 30° C. and shaken 15 s before and after readings. OD and GFP measurements were taken every 20 min. OD was measured at 600 nm (OD600). The GFP excitation was set at 395 nm with emission at 509 nm. The OD, RRE and MMF values determined for each of these sets of four wells at each time point were then averaged over this time window to create summary scores for that replicate. FIG. 4 shows the results of the evaluation of fidelity and efficiency of OTSs using the RFP-GFP fusion protein toolkit. Exemplary candidate tRNAs MG72, MG33, and MG24 have higher RRE and lower MMF compared to a prior known tRNA (F12).

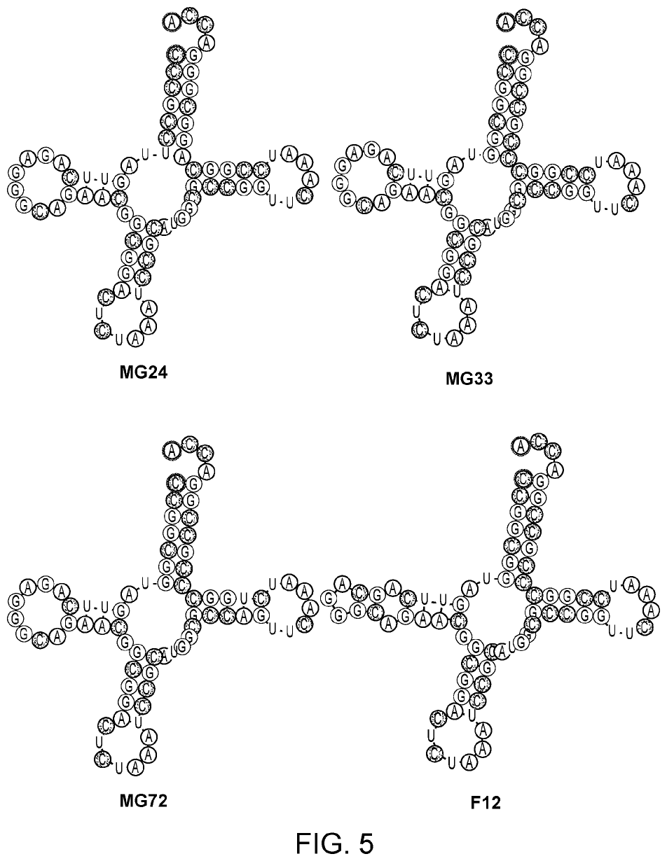

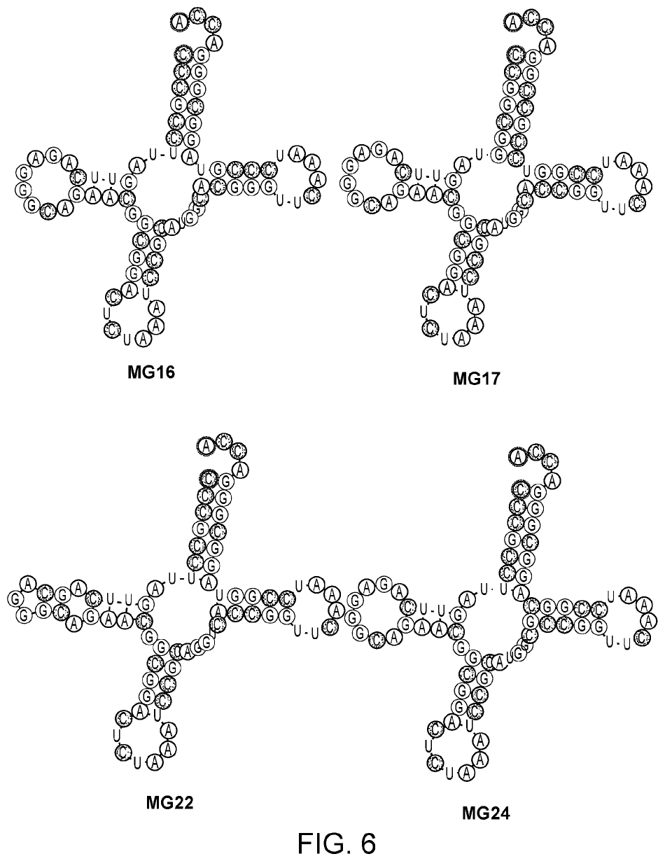

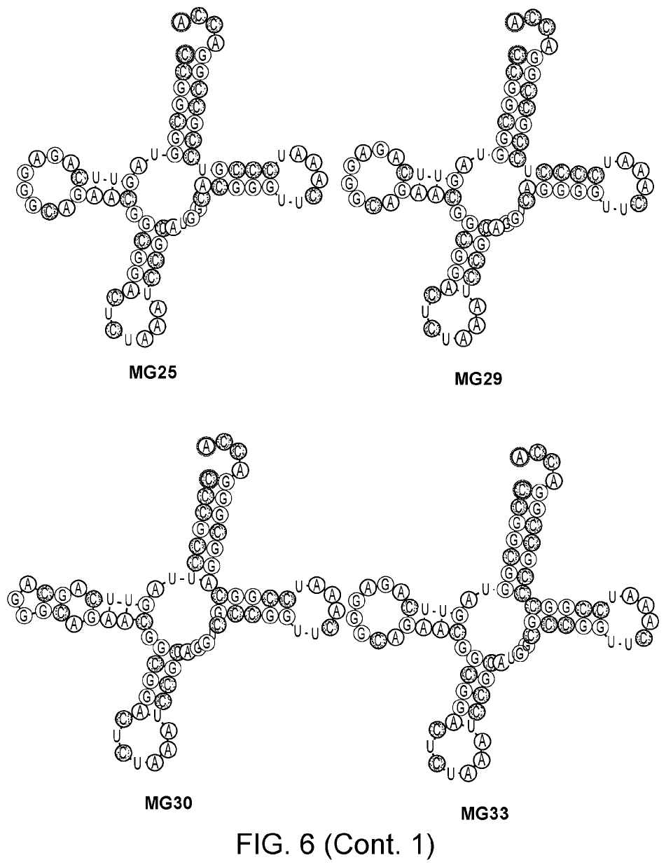

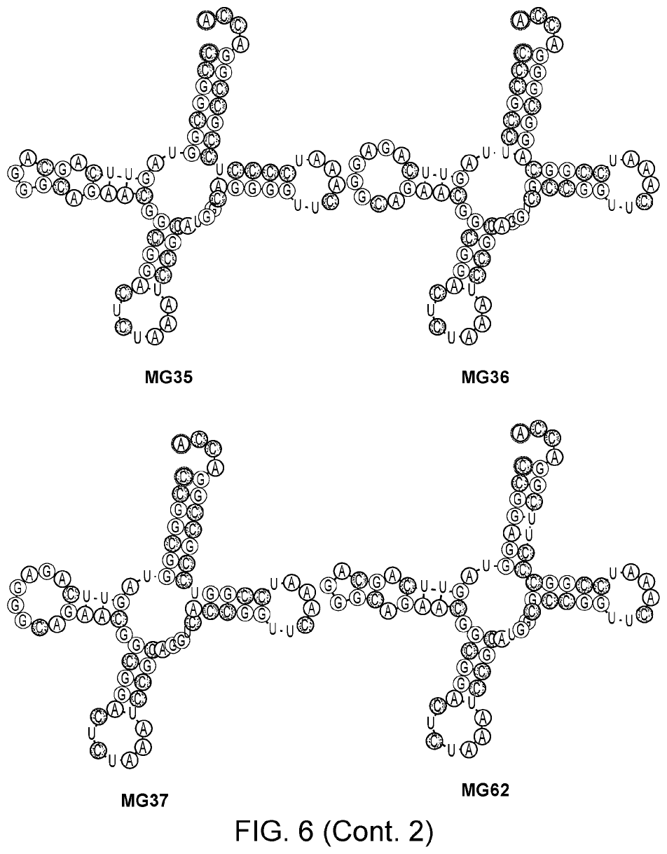

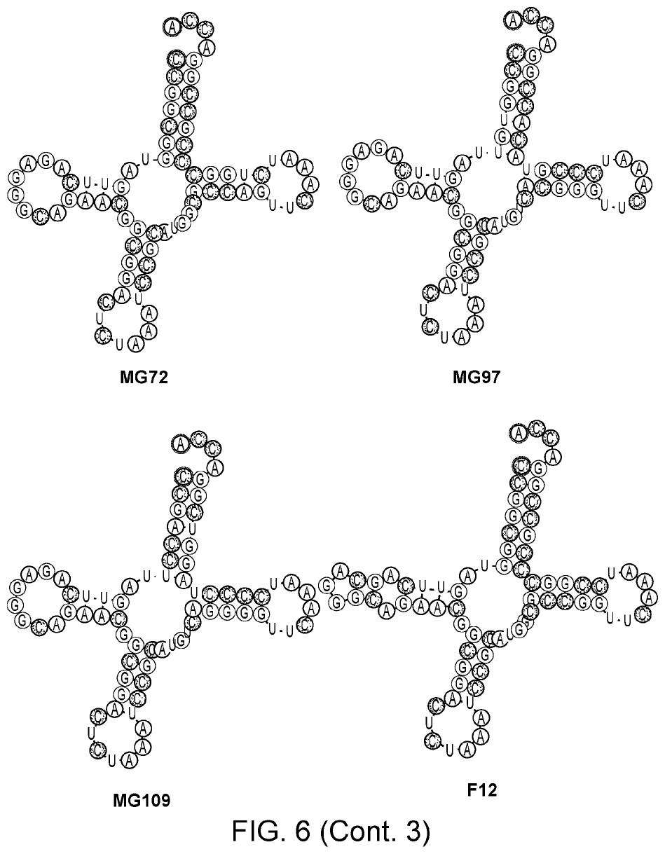

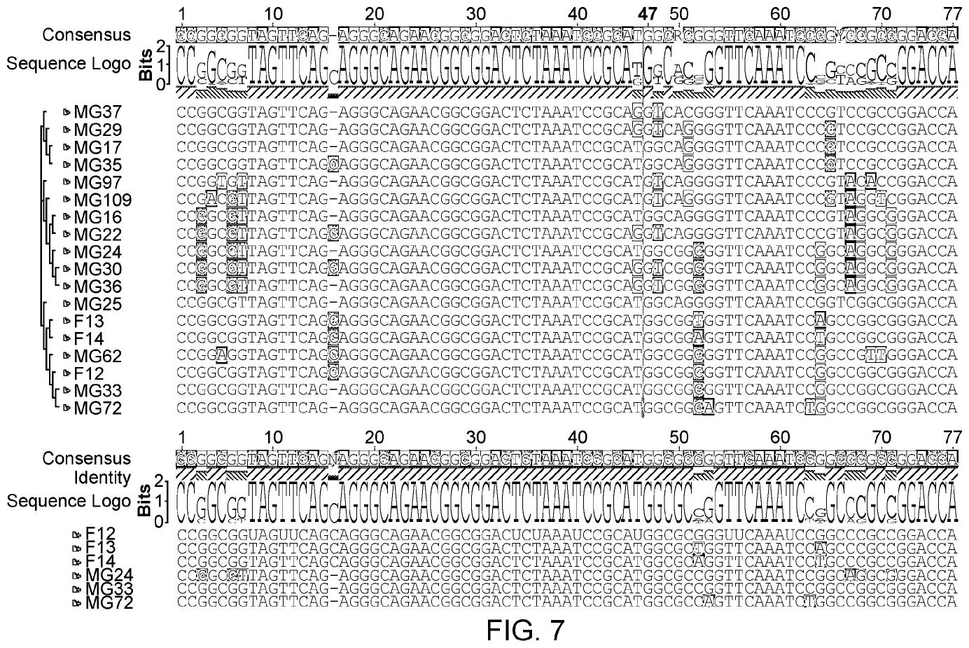

FIG. 5 and FIG. 6 show the cloverleaf structure and sequences of exemplary candidate tRNAs, including MG72, MG33, and MG24. FIG. 7 shows sequence alignments of the exemplary candidate tRNAs. These results show that several nucleic acid positions are altered in the D-loop, the stem of the T-loop, the stem of the acceptor stem and the variable loop compared to F12, F13 and F14 previously identified O-tRNA sequences. Compared to these previously identified tRNAs, the key structural differences identified in the candidate tRNAs included: a. in the D loop indel, i.e., C16 of the prior known O-tRNAs F12, F13, and F14, residing in the D loop of the tRNA, is deleted in candidates MG24, MG33, MG72, MG37, MG29, MG17, MG97, MG109, MG16, MG36, and MG25; b. different paired nucleotides residing in the stem of T loop, i.e., G53 and C63 in F12, F13, and F14 are mutated to A52 and T62 in candidate MG72; c. different paired nucleotides residing in the stem of T loop, i.e., C52 and G64 in F12, or T52 and A64 in F13, or A52 and T64 in F14 are mutated to G52 and C62 in candidates MG37, MG29, MG17, MG35, MG97, MG109, MG16, and MG22; d. Different paired nucleotides residing in the stem of T loop, i.e., C51 and G65 in F12, F13, and F14 are mutated to G51 and C65 in candidates MG29, MG17, MG35 and MG109; e. different paired nucleotides residing in the stem of acceptor stem, i.e., G3, G6, G7, C67, C68 and C71 in F12, F13, and F14 are mutated to C3, C6, T7, A66, G67 and G70 in candidates MG16, MG 22, MG24, MG30, MG36; f. different paired nucleotides residing in the stem of acceptor stem, i.e., C5, G7, C67 and G69 in F12, F13, and F14 are mutated to T5, T7, A67 and A69 in candidate MG97; e. different paired nucleotides residing in the stem of acceptor stem, i.e., G4, G6, G7, C67, C68 and C70 in F12, F13, and F14 are mutated to A4, C6, T7, A67, G68 and T70 in candidate MG109; g. different paired nucleotides residing in the stem of acceptor stem, i.e., C5, G69 and C70 in F12, F13, and F14 are mutated to A5, T69 and T70 in candidate MG62; h. different nucleotides residing in the variable loop, i.e., G48 in F12, F13, and F14 were mutated to T48 in candidate MG97; i. different nucleotides residing in the variable loop, i.e., C51 in F12, F13, and F14 were mutated to G51 in candidate MG17; j. different nucleotides residing in the variable loop, i.e., T46, G48 in F12, F13, and F14 were mutated to G46, T48 in candidates MG37, MG22, MG30 and MG36; k. different nucleotides residing in the variable loop, i.e., G48, C51 in F12, F13, and F14 were mutated to T48, G51 in candidate MG109; and 1. different nucleotides residing in the variable loop, i.e., T46, G48, C51 in F12, F13, and F14 were mutated to G46, T48, G51 in candidate MG29.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

INFORMAL SEQUENCE LISTING

| SEQ | | |
|---|---|---|
| ID | | |
| NO | DESCRIPTION | SEQUENCE |
| SEQ | F12 O-tRNA | CCGGCGGUAGUUCAGCAGGGCAGAACGGCGGACUCUAAAUC |
| ID | sequence | CGCAUGGCGCCGGUUCAAAUCCGGCCCGCCGGACCA |
| NO: 1 | | |
| SEQ | MG72 O-tRNA | CCGGCGGUAGUUCAGAGGGCAGAACGGCGGACUCUAAAUCC |
| ID | sequence | GCAUGGCGCCAGUUCAAAUCUGGCCCGCCGGACCA |
| NO: 2 | | |
| SEQ | MG24 O-tRNA | CCCGCCUUAGUUCAGAGGGCAGAACGGCGGACUCUAAAUCC |
| ID | sequence | GCAUGGCGCCGGUUCAAAUCCGGCAGGCGGGACCA |
| NO: 3 | | |
| SEQ | MG33 O-tRNA | CCGGCGGUAGUUCAGAGGGCAGAACGGCGGACUCUAAAUCC |
| ID | sequence | GCAUGGCGCCGGUUCAAAUCCGGCCCGCCGGACCA |
| NO: 4 | | |
| SEQ | MG16 O-tRNA | CCCGCCUUAGUUCAGAGGGCAGAACGGCGGACUCUAAAUCC |
| ID | sequence | GCAUGGCACGGGUUCAAAUCCCGUAGGCGGGACCA |
| NO: 5 | | |
| SEQ | MG17 O-tRNA | CCGGCGGUAGUUCAGAGGGCAGAACGGCGGACUCUAAAUCC |
| ID | sequence | GCAUGGCAGGGGUUCAAAUCCCCUCCGCCGGACCA |
| NO: 6 | | |
| SEQ | MG30 O-tRNA | CCCGCCUUAGUUCAGCAGGGCAGAACGGCGGACUCUAAAUC |
| ID | sequence | CGCAGGUCGCCGGUUCAAAUCCGGCAGGCGGGACCA |
| NO: 7 | | |
| SEQ | MG36 O-tRNA | CCCGCCUUAGUUCAGAGGGCAGAACGGCGGACUCUAAAUCC |
| ID | sequence | GCAGGUCGCCGGUUCAAAUCCGGCAGGCGGGACCA |
| NO: 8 | | |
| SEQ | MG22 O-tRNA | CCCGCCUUAGUUCAGCAGGGCAGAACGGCGGACUCUAAAUC |
| ID | sequence | CGCAGGUCACGGGUUCAAAUCCCGUAGGCGGGACCA |
| NO: 9 | | |
| SEQ | MG25 O-tRNA | CCGGCGGUAGUUCAGAGGGCAGAACGGCGGACUCUAAAUCC |
| ID | sequence | GCAUGGCACGGGUUCAAAUCCCGUCCGCCGGACCA |
| NO: 10 | | |
| SEQ | MG29 O-tRNA | CCGGCGGUAGUUCAGAGGGCAGAACGGCGGACUCUAAAUCC |
| ID | sequence | GCAGGUCAGGGGUUCAAAUCCCCUCCGCCGGACCA |
| NO: 11 | | |
| SEQ | MG35 O-tRNA | CCGGCGGUAGUUCAGCAGGGCAGAACGGCGGACUCUAAAUC |
| ID | sequence | CGCAUGGCAGGGGUUCAAAUCCCCUCCGCCGGACCA |
| NO: 12 | | |
| SEQ | MG37 O-tRNA | CCGGCGGUAGUUCAGAGGGCAGAACGGCGGACUCUAAAUCC |
| ID | sequence | GCAGGUCACGGGUUCAAAUCCCGUCCGCCGGACCA |
| NO: 13 | | |
| SEQ | MG62 O-tRNA | CCGGAGGUAGUUCAGCAGGGCAGAACGGCGGACUCUAAAUC |
| ID | sequence | CGCAUGGCGCCGGUUCAAAUCCGGCCCUUCGGACCA |
| NO: | | |
| 14 | | |
| SEQ | MG97 O-tRNA | CCGGUGUUAGUUCAGAGGGCAGAACGGCGGACUCUAAAUCC |
| ID | sequence | GCAUGUCACGGGUUCAAAUCCCGUACACCGGACCA |
| NO: | | |
| 15 | | |
| SEQ | MG109 O-tRNA | CCGACCUUAGUUCAGAGGGCAGAACGGCGGACUCUAAAUCC |
| ID | sequence | GCAUGUCAGGGGUUCAAAUCCCCUAGGUCGGACCA |
| NO: | | |
| 16 | | |
| SEQ | MG72 O-tRNA | CCGGCGGTAGTTCAGAGGGCAGAACGGCGGACTCTAAATCCG |
| ID | DNA sequence | CATGGCGCCAGTTCAAATCTGGCCCGCCGGACCA |
| NO: | | |
| 17 | | |
| SEQ | MG24 O-tRNA | CCCGCCTTAGTTCAGAGGGCAGAACGGCGGACTCTAAATCCG |
| ID | DNA sequence | CATGGCGCCGGTTCAAATCCGGCAGGCGGGACCA |
| NO: | | |
| 18 | | |
| SEQ | MG33 O-tRNA | CCGGCGGTAGTTCAGAGGGCAGAACGGCGGACTCTAAATCCG |
| ID | DNA sequence | CATGGCGCCGGTTCAAATCCGGCCCGCCGGACCA |
| NO: | | |
| 19 | | |
| SEQ | MG16 O-tRNA | CCCGCCTTAGTTCAGAGGGCAGAACGGCGGACTCTAAATCCG |
| ID | DNA sequence | CATGGCACGGGTTCAAATCCCGTAGGCGGGACCA |
| NO: | | |
| 20 | | |
| SEQ | MG17 O-tRNA | CCGGCGGTAGTTCAGAGGGCAGAACGGCGGACTCTAAATCCG |
| ID | DNA sequence | CATGGCAGGGGTTCAAATCCCCTCCGCCGGACCA |
| NO: | | |
| 21 | | |
| SEQ | MG30 O-tRNA | CCCGCCTTAGTTCAGCAGGGCAGAACGGCGGACTCTAAATCC |
| ID | DNA sequence | GCAGGTCGCCGGTTCAAATCCGGCAGGCGGGACCA |
| NO: | | |
| 22 | | |
| SEQ | MG36 O-tRNA | CCCGCCTTAGTTCAGAGGGCAGAACGGCGGACTCTAAATCCG |
| ID | DNA sequence | CAGGTCGCCGGTTCAAATCCGGCAGGCGGGACCA |
| NO: | | |
| 23 | | |
| SEQ | MG22 O-tRNA | CCCGCCTTAGTTCAGCAGGGCAGAACGGCGGACTCTAAATCC |
| ID | DNA sequence | GCAGGTCACGGGTTCAAATCCCGTAGGCGGGACCA |
| NO: | | |
| 24 | | |
| SEQ | MG25 O-tRNA | CCGGCGGTAGTTCAGAGGGCAGAACGGCGGACTCTAAATCCG |
| ID | DNA sequence | CATGGCACGGGTTCAAATCCCGTCCGCCGGACCA |
| NO: | | |
| 25 | | |
| SEQ | MG29 O-tRNA | CCGGCGGTAGTTCAGAGGGCAGAACGGCGGACTCTAAATCCG |
| ID | DNA sequence | CAGGTCAGGGGTTCAAATCCCCTCCGCCGGACCA |
| NO: | | |
| 26 | | |
| SEQ | MG35 O-tRNA | CCGGCGGTAGTTCAGCAGGGCAGAACGGCGGACTCTAAATCC |
| ID | DNA sequence | GCATGGCAGGGGTTCAAATCCCCTCCGCCGGACCA |
| NO: | | |
| 27 | | |
| SEQ | MG37 O-tRNA | CCGGCGGTAGTTCAGAGGGCAGAACGGCGGACTCTAAATCCG |
| ID | DNA sequence | CAGGTCACGGGTTCAAATCCCGTCCGCCGGACCA |
| NO: | | |
| 28 | | |
| SEQ | MG62 O-tRNA | CCGGAGGTAGTTCAGCAGGGCAGAACGGCGGACTCTAAATCC |
| ID | DNA sequence | GCATGGCGCCGGTTCAAATCCGGCCCTTCGGACCA |
| NO: | | |
| 29 | | |
| SEQ | MG97 O-tRNA | CCGGTGTTAGTTCAGAGGGCAGAACGGCGGACTCTAAATCCG |
| ID | DNA sequence | CATGTCACGGGTTCAAATCCCGTACACCGGACCA |
| NO: | | |
| 30 | | |
| SEQ | MG109 O-tRNA | CCGACCTTAGTTCAGAGGGCAGAACGGCGGACTCTAAATCCG |
| ID | DNA sequence | CATGTCAGGGGTTCAAATCCCCTAGGTCGGACCA |
| NO: | | |
| 31 | | |
| SEQ | Wild type Mj | CCGGCGGUAGUUCAGCCUGGUAGAACGGCGGACUGUAGAUC |
| ID | tyrosyl-tRNA | CGCAUGUCGCUGGUUCAAAUCCGGCCCGCCGGACCA |
| NO: | | |
| 32 | | |
| SEQ | Mj tyrosyl-tRNA | CCGGCGGTAGTTCAGCCTGGTAGAACGGCGGACTGTAGATCC |
| ID | Wild type | GCATGTCGCTGGTTCAAATCCGGCCCGCCGGACCA |
| NO: | DNA sequence | |
| 33 | | |
| SEQ | Library tRNA | CNNGCNNTAGTTCAGCAGGGCAGAACGGCGGACTCTAAATCC |
| ID | | GCATGGCWNRGGTTCAAATCCYNWNNGCNNGACCA |
| NO: | | |
| 34 | | |
| SEQ | Mj tyrosyl-tRNA | MDEFEMIKRNTSEIISEEELREVLKKDEKSAVIGFEPSGKIHLGHY |
| ID | synthetase “E9 | LQIKKMIDLQNAGFDIIIYLADLHAYLNQKGELDEIRKIGDYNKK |
| NO: | (D286R)” | VFEAMGLKAKYVYGSEHGLDKDYTLNVYRLALKTTLKRARRS |
| 35 | amino acid | MELIAREDENPKVAEVIYPIMQVNGIHYEGVDVAVGGMEQRKIH |
| | sequence | MLARELLPKKVVCIHNPVLTGLDGEGKMSSSKGNFIAVDDSPEEI |
| | | RAKIKKAYCPAGVVEGNPIMEIAKYFLEYPLTIKRPEKFGGDLTV |
| | | NSYEELESLFKNKELHPMRLKNAVAEELIKILEPIRKRL |
| SEQ | O-tRNA | CCX₁X₂X₃X₄X₅UAGUUCAGX₆AGGGCAGAACGGCGGACUCUAA |
| ID | consensus | AUCCGCAX₇GX₈CX₉X₁₀X₁₁X₁₂GUCAAAUCX₁₃X₁₄X₁₅X₁₆X₁₇X₁₈X₁₉ |
| NO: | sequence | X₂₀X₂₁GGACCA; |
| 36 | | wherein: |
| | | X₁ is C or G; |
| | | X₂ is A or G; |
| | | X₃ is A, U or C; |
| | | X₄ is C or G; |
| | | X₅ is U or G; |
| | | X₆ is C or del; |
| | | X₇ is G or U; |
| | | X₈ is G or U; |
| | | X₉ is A or G; |
| | | X₁₀ is G or C; |
| | | X₁₁ is G, C, A or U; |
| | | X₁₂ is G or A; |
| | | X₁₃ is C or U; |
| | | X₁₄ is G, C, A or U; |
| | | X₁₅ is G or C; |
| | | X₁₆ is U or C; |
| | | X₁₇ is A or C; |
| | | X₁₈ is G or C; |
| | | X₁₉ isA, G or U; |
| | | X₂₀ is U or C; |
| | | X₂₁ is C or G; |
| SEQ | F13 | CCGGCGGUAGUUCAGCAGGGCAGAACGGCGGACUCUAAAUC |
| ID | | CGCAUGGCGCUGGUUCAAAUCCAGCCCGCCGGACCA |
| NO: | | |
| 37 | | |
| SEQ | F14 | CCGGCGGUAGUUCAGCAGGGCAGAACGGCGGACUCUAAAUC |
| ID | | CGCAUGGCGCAGGUUCAAAUCCUGCCCGCCGGACCA |
| NO: | | |
| 38 | | |
| SEQ | Mj tyrosyl-tRNA | MDEFEMIKRNTSEIVSEEELREVLKKDEKSAVIGFEPSGKIHLGHY |
| ID | synthetase “E9 | LQIKKMIDLQNAGFDIIIYLADLHAYLNQKGELDEIRKIGDYNKK |
| NO: | (E9VR)” | VFEAMGLKAKYVYGSEHGLDKDYTLNVYRLALKTTLKRARRS |
| 39 | amino acid | MELIAREDENPKVAEVIYPIMQVNGIHYEGVDVAVGGMEQRKIH |
| | sequence; has | MLARELLPKKVVCIHNPVLTGLDGEGKMSSSKGNFIAVDDSPEEI |
| | I15V and | RAKIKKAYCPAGVVEGNPIMEIAKYFLEYPLTIKRPEKFGGDLTV |
| | D286R | NSYEELESLFKNKELHPMRLKNAVAEELIKILEPIRKRL |
| | mutations | |

1. An orthogonal tRNA (O-tRNA) comprising a nucleic acid sequence at least 85% identical to the sequence set forth in SEQ ID NO: 1 and comprising a deletion of the cytosine located at nucleic acid position 16 of the O-tRNA, wherein the nucleic acid positions correspond to the sequence set forth in SEQ ID NO: 1; and wherein the O-tRNA is capable of being aminoacylated with at least one non-standard amino acid (nsAA) by an orthogonal aminoacyl tRNA synthetase (O-RS).

2. The O-tRNA of claim 1, wherein the nucleic acid sequence is at least 90% identical, at least 91% identical, at least 92% identical, at least 93% identical, at least 94% identical, at least 95% identical, at least 96% identical, at least 97% identical, at least 98% identical, or at least 98.7% identical to the sequence set forth in SEQ ID NO: 1.

3. The O-tRNA of claim 1 or 2, wherein the O-tRNA comprises an adenine at nucleic acid position 53 and a uracil at nucleic acid position 63, wherein the nucleic acid positions correspond to the sequence set forth in SEQ ID NO:1.

4. The O-tRNA of claim 3, wherein the O-tRNA comprises a nucleic acid sequence consisting of the sequence set forth in SEQ ID NO: 2.

5. The O-tRNA of claim 1 or 2, wherein the O-tRNA comprises a cytosine at nucleic acid positions 3 and 6; a uracil at nucleic acid position 7, an adenosine at nucleic acid position 67, and a guanine at nucleic acid positions 68 and 71, wherein the nucleic acid positions correspond to the sequence set forth in SEQ ID NO: 1.

6. The O-tRNA of claim 5, wherein the O-tRNA comprises a nucleic acid sequence consisting of the sequence set forth in SEQ ID NO: 3.

7. The O-tRNA of claim 1, wherein the O-tRNA comprises a nucleic acid sequence consisting of the sequence set forth in SEQ ID NO: 4.

8. The O-tRNA of claim 1 or 2, wherein the O-tRNA comprises the sequence CAG-AGGGCAG at nucleic acid positions 13 to 23, wherein the nucleic acid positions correspond to the sequence set forth in SEQ ID NO: 1.

9. An O-tRNA comprising a nucleic acid sequence consisting of a sequence set forth in SEQ ID NO: 36, wherein the sequence does not comprise SEQ ID NO: 1, SEQ ID NO: 37, or SEQ ID NO: 38.

10. The O-tRNA of claim 9, wherein the O-tRNA comprises a cytosine at position 3.

11. The O-tRNA of claim 9 or 10, wherein the O-tRNA comprises an adenine at position 4.

12. The O-tRNA of any one of claims 9-11, wherein the O-tRNA comprises a uracil at position 5.

13. The O-tRNA of any one of claims 9-12, wherein the O-tRNA comprises a cytosine at position 6.

14. The O-tRNA of any one of claims 9-13, wherein the O-tRNA comprises a uracil at position 7.

15. The O-tRNA of any one of claims 9-14, wherein the O-tRNA comprises a guanine at position 46.

16. The O-tRNA of any one of claims 9-15, wherein the O-tRNA comprises a uracil at position 48.

17. The O-tRNA of any one of claims 9-16, wherein the O-tRNA comprises an adenine at position 50.

18. The O-tRNA of any one of claims 9-17, wherein the O-tRNA comprises a guanine at position 51.

19. The O-tRNA of any one of claims 9-18, wherein the O-tRNA comprises an adenine at position 53.

20. The O-tRNA of any one of claims 9-19, wherein the O-tRNA comprises a uracil at position 63.

21. The O-tRNA of any one of claims 9-20, wherein the O-tRNA comprises a cytosine at position 65.

22. The O-tRNA of any one of claims 9-21, wherein the O-tRNA comprises a uracil at position 66.

23. The O-tRNA of any one of claims 9-22, wherein the O-tRNA comprises an adenine at position 67.

24. The O-tRNA of any one of claims 9-23, wherein the O-tRNA comprises a guanine at position 68.

25. The O-tRNA of any one of claims 9-24, wherein the O-tRNA comprises an adenine or a uracil at position 69.

26. The O-tRNA of any one of claims 9-25, wherein the O-tRNA comprises a uracil at position 70.

27. The O-tRNA of any one of claims 9-26, wherein the O-tRNA comprises a guanine at position 71.

28. The O-tRNA of any one of claims 9-27, wherein the O-tRNA comprises a cytosine at position 3, a cytosine at position 6, and a uracil at position 7.

29. The O-tRNA of any one of claims 9-28, wherein the O-tRNA comprises a guanine at position 46 and a uracil at position 48.

30. The O-tRNA of any one of claims 9-29, wherein the O-tRNA comprises an adenine at position 67, a guanine at position 68, and a guanine at position 71.

31. An O-tRNA comprising a nucleic acid sequence consisting of a sequence set forth in any one of SEQ ID NOs: 2-16.

32. The O-tRNA of any one of claims 1-31, wherein the O-tRNA is aminoacylated.

33. The O-tRNA of claim 32, wherein the O-tRNA is aminoacylated with the nsAA.

34. The O-tRNA of claim 33, wherein the nsAA has the structure according to Formula I; wherein the R group is any substituent other than a corresponding substituent used in the twenty natural amino acids.

35. The O-tRNA of claim 34, wherein the nsAA has the structure according to Formula I wherein the R group comprises an alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amine, or combinations thereof.

36. The O-tRNA of any one of claims 33-35, wherein the nsAA is selected from the group consisting of: an amino acid comprising a photoactivatable cross-linker, a spin-labeled amino acid, a fluorescent amino acid, a metal binding amino acid, a metal containing amino acid, a radioactive amino acid, an amino acid comprising at least one novel functional group, an amino acid that covalently or noncovalently interacts with other molecules, a photocaged amino acid, a photoisomerizable amino acid, an amino acids comprising biotin or a biotin analogue, a carbohydrate-modified amino acid, an amino acid comprising polyethylene glycol or polyether, a heavy atom substituted amino acid, a chemically cleavable amino acid, a photocleavable amino acid, and combinations thereof.

37. The O-tRNA of any one of claims 33-36, wherein the nsAA comprises a tyrosine analog.

38. The O-tRNA of claim 37, wherein the tyrosine analog is selected from the group consisting of a para-substituted tyrosine, an ortho-substituted tyrosine, and a meta-substituted tyrosine.

39. The O-tRNA of claim 38, wherein the substituted tyrosine comprises a keto group, an acetyl group, a benzoyl group, an amino group, a hydrazine, a hydroxyamine, a thiol group, a carboxy group, an isopropyl group, a methyl group, a branched hydrocarbon, a saturated or unsaturated hydrocarbon, an O-methyl group, a polyether group, a nitro group, or combinations thereof.

40. The O-tRNA of any one of claims 33-36, wherein the nsAA comprises a glutamine analog.

41. The O-tRNA of claim 40, wherein the glutamine analog comprises an α-hydroxy derivative, a γ-substituted derivative, a cyclic derivative, or an amide substituted glutamine derivative.

42. The O-tRNA of any one of claims 33-36, wherein the nsAA comprises a phenylalanine analog.

43. The O-tRNA of claim 41, wherein the phenylalanine analog is an amino-, an isopropyl-, or an O-allyl-containing phenylalanine analog.

44. The O-tRNA of claim 42 or 43, wherein the phenylalanine analog is selected from the group consisting of a para-substituted phenylalanine, an ortho-substituted phenylalanine, and a meta-substituted phenylalanine.

45. The O-tRNA of claim 44, wherein the substituent comprises a hydroxy group, a methoxy group, a methyl group, an allyl group, an aldehyde, an azido, an iodo, a bromo, a keto group or an acetyl group.

46. The O-tRNA of claim 45, wherein the nsAA comprises a para-acetyl phenylalanine.

47. The O-tRNA of any one of claims 33-36, wherein the nsAA is selected from the group consisting of a p-propargyl phenylalanine, an O-methyl-L-tyrosine, an L-3-(2-naphthyl)alanine, a 3-methylphenylalanine, an O-4-allyl-L-tyrosine, a 4-propyl-L-tyrosine, a tri-O-acetyl-GlcNAcB-serine, an L-Dopa, a fluorinated phenylalanine, an isopropyl-L-phenylalanine, a p-acyl-L-phenylalanine, a p-benzoyl-L-phenylalanine, an L-phosphoserine, a phosphono serine, a phosphonotyrosine, a p-iodo-phenylalanine, a p-bromophenylalanine, a p-amino-L-phenylalanine, and an isopropyl-L-phenylalanine.

48. The O-tRNA of any one of claims 33-36, wherein the nsAA is selected from the group consisting of 4-acetyl-phenylalanine (AcF), 4-azido-phenylalanine (AzF); 4-propargyloxyphenylalanine (PaF); and 4-aminophenylalanine (AmF).

49. The O-tRNA of claim 48, wherein the nsAA comprises an 4-acetyl-phenylalanine (AcF).

50. The O-tRNA of claim 48, wherein the nsAA comprises an 4-azido-phenylalanine (AzF).

51. The O-tRNA of claim 48, wherein the nsAA comprises an 4-propargyloxyphenylalanine (PaF).

52. The O-tRNA of claim 48, wherein the nsAA comprises an 4-aminophenylalanine (AmF).

53. The O-tRNA of any one of claims 32-34, wherein the O-tRNA is chemically aminoacylated.

54. The O-tRNA of any one of claims 32-34, wherein the O-tRNA is enzymatically aminoacylated.

55. The O-tRNA of claim 54, wherein the O-tRNA is enzymatically aminoacylated by a ribozyme.

56. The O-tRNA of any one of claims 1-55, wherein the O-tRNA is derived from archaeal tRNA.

57. The O-tRNA of claim 56, wherein the O-tRNA is derived from M. jannaschii.

58. An orthogonal tRNA synthetase (O-RS) comprising the amino acid sequence consisting of the sequence set forth in SEQ ID NO: 39.

59. An orthogonal translation system (OTS) comprising the O-tRNA of any one of claims 1-57 and an O-RS.

60. The OTS of claim 59, wherein the O-RS comprises an O-RS of an M. jannaschii. tyrosyl-tRNA synthetase.

61. The OTS of claim 60, wherein the O-RS comprises an amino acid sequence consisting of the sequence set forth in SEQ ID NO: 35 or 39.

62. The OTS of claims 59 or 60, further comprising the nsAA.

63. The OTS of any one of claims 59-62, wherein the nsAA has the structure according to Formula I; wherein the R group is any substituent other than a corresponding substituent used in the twenty natural amino acids.

64. The OTS of claim 62 or 63, wherein the nsAA has the structure according to Formula I wherein the R group comprises an alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amine, or combinations thereof.

65. The OTS of any one of claims 62-64, wherein the nsAA is selected from the group consisting of: an amino acid comprising a photoactivatable cross-linker, a spin-labeled amino acid, a fluorescent amino acid, a metal binding amino acid, a metal containing amino acid, a radioactive amino acid, an amino acid comprising at least one novel functional group, an amino acid that covalently or noncovalently interacts with other molecules, a photocaged amino acid, a photoisomerizable amino acid, an amino acids comprising biotin or a biotin analogue, a carbohydrate-modified amino acid, an amino acid comprising polyethylene glycol or polyether, a heavy atom substituted amino acid, a chemically cleavable amino acid, a photocleavable amino acid, and combinations thereof.

66. The OTS of any one of claims 62-65, wherein the nsAA comprises a tyrosine analog.

67. The OTS of claim 66, wherein the tyrosine analog is selected from the group consisting of a para-substituted tyrosine, an ortho-substituted tyrosine, and a meta-substituted tyrosine.

68. The OTS of claim 67, wherein the substituted tyrosine comprises a keto group, an acetyl group, a benzoyl group, an amino group, a hydrazine, a hydroxyamine, a thiol group, a carboxy group, an isopropyl group, a methyl group, a branched hydrocarbon, a saturated or unsaturated hydrocarbon, an O-methyl group, a polyether group, a nitro group, or combinations thereof.

69. The OTS of any one of claims 62-65, wherein the nsAA comprises a glutamine analog.

70. The OTS of claim 69, wherein the glutamine analog comprises an α-hydroxy derivative, a γ-substituted derivative, a cyclic derivative, or an amide substituted glutamine derivative.

71. The OTS of any one of claims 62-65, wherein the nsAA comprises a phenylalanine analog.

72. The OTS of claim 71, wherein the phenylalanine analog is an amino-, an isopropyl-, or an O-allyl-containing phenylalanine analog.

73. The OTS of claim 71 or 72, wherein the phenylalanine analog is selected from the group consisting of a para-substituted phenylalanine, an ortho-substituted phenylalanine, and a meta-substituted phenylalanine.

74. The OTS of claim 73, wherein the substituent comprises a hydroxy group, a methoxy group, a methyl group, an allyl group, an aldehyde, an azido, an iodo, a bromo, a keto group or an acetyl group.

75. The OTS of claim 74, wherein the nsAA comprises a para-acetyl phenylalanine.

76. The OTS of any one of claims 62-65, wherein the nsAA is selected from the group consisting of a p-propargyl phenylalanine, an O-methyl-L-tyrosine, an L-3-(2-naphthyl)alanine, a 3-methylphenylalanine, an O-4-allyl-L-tyrosine, a 4-propyl-L-tyrosine, a tri-O-acetyl-GlcNAcB-serine, an L-Dopa, a fluorinated phenylalanine, an isopropyl-L-phenylalanine, a p-acyl-L-phenylalanine, a p-benzoyl-L-phenylalanine, an L-phosphoserine, a phosphono serine, a phosphonotyrosine, a p-iodo-phenylalanine, a p-bromophenylalanine, a p-amino-L-phenylalanine, and an isopropyl-L-phenylalanine.

77. The OTS of claim 76, wherein the nsAA comprises an O-methyl-L-tyrosine.

78. The OTS of claim 76, wherein the nsAA comprises an L-3-(2-naphthyl)alanine.

79. The OTS of any one of claims 59-78, wherein the O-tRNA recognizes a selector codon.

80. The OTS of claim 79, wherein the selector codon is an amber codon.

81. The OTS of claim 79 or 80, further comprising a polynucleotide comprising at least one selector codon that is recognized by the O-tRNA.

82. The OTS of any one of claims 59-81, further comprising a mutant EF-Tu.

83. The OTS of any one of claims 59-82, wherein the OTS is a cell-free translation system.

84. The OTS of claim 83, wherein the cell-free translation system is a cell lysate.

85. The OTS of claim 83, wherein the cell-free translation system is a reconstituted system.

86. The OTS of any one of claims 59-82, wherein the OTS is a cellular translation system.

87. A cell comprising the OTS of any one of claims 59-86.

88. The cell of claim 87, wherein the cell is a non-eukaryotic cell or a prokaryotic cell.

89. The cell of claim 88, wherein the prokaryotic cell is Escherichia coli.

90. The cell of claim 84, wherein the cell is a eukaryotic cell.

91. The cell of claim 90, wherein the cell is a yeast cell.

92. The cell of claim 90, wherein the cell is a fungal cell.

93. The cell of claim 90, wherein the cell is a mammalian cell.

94. The cell of claim 90, wherein the cell is an insect cell.

95. The cell of claim 90, wherein the cell is a plant cell.

96. The cell of any one of claims 84-90, wherein the cell encodes a mutation in an EF-Tu.

97. The cell of any one of claims 84-96, wherein the cell has reduced expression of Release Factor 1 compared to an otherwise identical wild-type cell.

98. A polypeptide comprising at least one nsAA, wherein the polypeptide is produced by the OTS of any one of claims 59-81 or the cell of any one of claims 84-90.

99. The polypeptide of claim 98, wherein the polypeptide comprises an antibody or antigen binding fragment thereof.

100. The polypeptide of claim 98, wherein the polypeptide comprises human growth hormone.

101. A polynucleotide comprising a nucleic acid sequence of an O-tRNA comprising a nucleic acid sequence consisting of a sequence set forth in any one of SEQ ID NOs: 2-31.

102. The polynucleotide of claim 101, further comprising a nucleic acid sequence complementary to the O-tRNA sequence consisting of a sequence set forth in any one of SEQ ID NOs: 2-31.

103. A polynucleotide comprising a nucleic acid sequence encoding an O-RS comprising an amino acid sequence consisting of a sequence set forth in SEQ ID NO: 39.

104. The polynucleotide of claim 103, further comprising a nucleic acid sequence complementary to the O-RS comprising a nucleic acid sequence consisting of the sequence set forth in SEQ ID NO: 39.

105. A polynucleotide or set of polynucleotides comprising a nucleic acid sequence of an O-tRNA comprising a nucleic acid sequence set forth in any one of SEQ ID NOs: 2-31 and a nucleic acid sequence encoding an O-RS of an M. jannaschii. tyrosyl-tRNA.

106. The polynucleotide or set of polynucleotides of claim 105, wherein the O-RS comprises an amino acid sequence consisting of the sequence set forth in SEQ ID NO: 35 or 39.

107. A vector comprising at least one polynucleotide of any one of claims 101-105.

108. The vector of claim 107, wherein the vector is an expression vector.

109. The vector of claim 108, wherein the vector is selected from the group consisting of a plasmid, a cosmid, a phage, and a virus.

110. A cell comprising the polynucleotide of any one of claims 101-105 or the vector of any one of claims 107-109.

111. A kit comprising one or more of the polynucleotide(s) of any of claims 101-105, the vector of any one of claims 107-109, or the cell of any one of claims 84-90 or 110, and instructions for use.

112. A method of producing a polypeptide comprising at least one nsAA, comprising expressing in a cell an O-tRNA comprising a nucleic acid sequence at least 85% identical to the sequence set forth in SEQ ID NO: 1 and comprising a deletion of the cytosine located at nucleic acid position 16 of the O-tRNA, wherein the nucleic acid positions correspond to the sequence set forth in SEQ ID NO: 1; and wherein the O-tRNA is capable of being aminoacylated with the at least one non-standard amino acid (nsAA) by an O-RS.

113. The method of claim 112, wherein the nucleic acid sequence is at least 90% identical, at least 91% identical, at least 92% identical, at least 93% identical, at least 94% identical, at least 95% identical, at least 96% identical, at least 97% identical, at least 98% identical, or at least 98.7% identical to the sequence set forth in SEQ ID NO: 1.

114. The method of claim 112 or 113, wherein the O-tRNA comprises an adenine at nucleic acid position 53 and a uracil at nucleic acid position 63, wherein the nucleic acid positions correspond to the sequence set forth in SEQ ID NO: 1.

115. The method of claim 114, wherein the O-tRNA comprises a nucleic acid sequence consisting of the sequence set forth in SEQ ID NO: 2.

116. The method of claim 112 or 113, wherein the O-tRNA comprises a cytosine at nucleic acid positions 3 and 6; a uracil at nucleic acid position 7, an adenosine at nucleic acid position 67, and a guanine at nucleic acid positions 68 and 71, wherein the nucleic acid positions correspond to the sequence set forth in SEQ ID NO: 1.

117. The method of claim 116, wherein the O-tRNA comprises a nucleic acid sequence consisting of the sequence set forth in SEQ ID NO: 3.

118. The method of claim 112, wherein the O-tRNA comprises a nucleic acid sequence set forth in SEQ ID NO: 4.

119. The method of claim 112 or 113, wherein the O-tRNA comprises the sequence CAG-AGGGCAG at nucleic acid positions 13 to 23, wherein the nucleic acid positions correspond to the sequence set forth in SEQ ID NO: 1.

120. A method of producing a polypeptide comprising at least one nsAA, comprising expressing in a cell an O-tRNA comprising a nucleic acid sequence consisting of a sequence set forth in SEQ ID NO: 36, wherein the sequence does not comprise SEQ ID NO: 1, SEQ ID NO: 37, or SEQ ID NO: 38.

121. The method of claim 118, wherein the O-tRNA comprises a cytosine at position 3.

122. The method of claim 118 or 119, wherein the O-tRNA comprises an adenine at position 4.

123. The method of any one of claims 118-120, wherein the O-tRNA comprises a uracil at position 5.

124. The method of any one of claims 118-121, wherein the O-tRNA comprises a cytosine at position 6.

125. The method of any one of claims 118-122, wherein the O-tRNA comprises a uracil at position 7.

126. The method of any one of claims 118-123, wherein the O-tRNA comprises a guanine at position 46.

127. The method of any one of claims 118-124, wherein the O-tRNA comprises a uracil at position 48.

128. The method of any one of claims 118-125, wherein the O-tRNA comprises an adenine at position 50.

129. The method of any one of claims 118-126, wherein the O-tRNA comprises a guanine at position 51.

130. The method of any one of claims 118-127, wherein the O-tRNA comprises an adenine at position 53.

131. The method of any one of claims 118-128, wherein the O-tRNA comprises a uracil at position 63.

132. The O-tRNA of any one of claims 118-129, wherein the O-tRNA comprises a cytosine at position 65.

133. The method of any one of claims 118-130, wherein the O-tRNA comprises a uracil at position 66.

134. The method of any one of claims 118-131, wherein the O-tRNA comprises an adenine at position 67.

135. The method of any one of claims 118-132, wherein the O-tRNA comprises a guanine at position 68.

136. The method of any one of claims 118-133, wherein the O-tRNA comprises an adenine or a uracil at position 69.

137. The method of any one of claims 118-134, wherein the O-tRNA comprises a uracil at position 70.

138. The method of any one of claims 118-135, wherein the O-tRNA comprises a guanine at position 71.

139. The method of any one of claims 118-136, wherein the O-tRNA comprises a cytosine at position 3, a cytosine at position 6, and a uracil at position 7.

140. The method of any one of claims 118-137, wherein the O-tRNA comprises a guanine at position 46 and a uracil at position 48.

141. The method of any one of claims 118-138, wherein the O-tRNA comprises an adenine at position 67, a guanine at position 68, and a guanine at position 71.

142. The method of any of claims 118-139, wherein the O-tRNA comprises a nucleic acid sequence consisting of the sequence set forth in any one of SEQ ID NOs: 2-16.

143. The method of any one of claims 112-140, further comprising expressing an O-RS in the cell.

144. The method of any one of claims 112-141, wherein the O-RS is an O-RS of an M. jannaschii tyrosyl-tRNA synthetase.

145. The method of claim 144, wherein the O-RS comprises an amino acid sequence consisting of the sequence set forth in SEQ ID NO: 35 or 39.

146. The method of claim 142, wherein the O-tRNA comprises a nucleic acid sequence set forth in any one of SEQ ID NOs: 2-16 and the O-RS comprises an amino acid sequence consisting of the sequence set forth in SEQ ID NO: 35 or 39.

147. The method of any one of claims 112-146, wherein the O-RS aminoacylates the O-tRNA with the nsAA.

148. The method of any one of claims 142-147, wherein the nsAA has the structure according to Formula I; and wherein the R group is any substituent other than a corresponding substituent used in the twenty natural amino acids.

149. The method of claim 148, wherein the nsAA has the structure according to Formula I; and wherein the R group comprises an alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amine, or combinations thereof.

150. The method of any one of claims 110-149, wherein the nsAA is selected from the group consisting of an amino acid comprising a photoactivatable cross-linker, a spin-labeled amino acid, a fluorescent amino acid, a metal binding amino acid, a metal containing amino acid, a radioactive amino acid, an amino acid with at least one novel functional group, an amino acid that covalently or noncovalently interacts with other molecules, a photocaged amino acid, a photoisomerizable amino acid, an amino acid comprising biotin or a biotin analogue, a carbohydrate-modified amino acid, and an amino acid comprising polyethylene glycol or polyether, a heavy atom substituted amino acid, a chemically cleavable amino acid, a photocleavable amino acid, and combinations thereof.

151. The method of any one of claims 110-150, wherein the nsAA comprises a tyrosine analog.

152. The method of claim 151, wherein the tyrosine analog is selected from the group consisting of a para-substituted tyrosine, an ortho-substituted tyrosine, and a meta-substituted tyrosine.

153. The method of claim 152, wherein the substituted tyrosine comprises a keto group, an acetyl group, a benzoyl group, an amino group, a hydrazine, a hydroxyamine, a thiol group, a carboxy group, an isopropyl group, a methyl group, a branched hydrocarbon, a saturated or unsaturated hydrocarbon, an O-methyl group, a polyether group, a nitro group, or combinations thereof.

154. The method of any one of claims 110-150, wherein the nsAA comprises a glutamine analog.

155. The method of claim 154, wherein the glutamine analog comprises a α-hydroxy derivative, a T-substituted derivative, a cyclic derivative, or an amide substituted glutamine derivative.

156. The method of any one of claims 110-150, wherein the nsAA comprises a phenylalanine analog.

157. The method of claim 153, wherein the phenylalanine analog is an amino-, an isopropyl-, or a O-allyl-containing phenylalanine analog.

158. The method of claim 156 or 157, wherein the phenylalanine analog is selected from the group consisting of a para-substituted phenylalanine, an ortho-substituted phenylalanine, and a meta-substituted phenylalanine.

159. The method of any one of claims 110-158, wherein the substituent comprises a hydroxy group, a methoxy group, a methyl group, an allyl group, an aldehyde, an azido, an iodo, a bromo, a keto group or an acetyl group.

160. The method of claim 159, wherein the nsAA comprises a para-acetyl phenylalanine.

161. The method of any one of claims 110-150, wherein the nsAA is selected from the group consisting of a p-propargyl phenylalanine, a O-methyl-L-tyrosine, an L-3-(2-naphthyl)alanine, a 3-methylphenylalanine, an O-4-allyl-L-tyrosine, a 4-propyl-L-tyrosine, a tri-O-acetyl-GlcNAcB-serine, an L-Dopa, a fluorinated phenylalanine, an isopropyl-L-phenylalanine, a p-acyl-L-phenylalanine, a p-benzoyl-L-phenylalanine, an L-phosphoserine, a phosphono serine, a phosphonotyrosine, a p-iodo-phenylalanine, a p-bromophenylalanine, a p-amino-L-phenylalanine, and an isopropyl-L-phenylalanine.

162. The O-tRNA of any one of claims 142-150, wherein the nsAA is selected from the group consisting of 4-acetyl-phenylalanine (AcF), 4-azido-phenylalanine (AzF); 4-propargyloxyphenylalanine (PaF); and 4-aminophenylalanine (AmF).

163. The method of claim 162, wherein the nsAA comprises an 4-acetyl-phenylalanine (AcF).

164. The method of claim 162, wherein the nsAA comprises an 4-azido-phenylalanine (AzF).

165. The method of claim 162, wherein the nsAA comprises an 4-propargyloxyphenylalanine (PaF).

166. The method of claim 162, wherein the nsAA comprises an 4-aminophenylalanine (AmF).

167. The method of any one of claims 110-166, wherein the nsAA is biosynthesized by the cell.

168. The method of any one of claims 110-166, wherein the nsAA is provided to the cell exogenously.

169. The method of any one of claims 142-168, wherein the cell is a non-eukaryotic cell or a prokaryotic cell.

170. The method of claim 169, wherein the prokaryotic cell is Escherichia coli.

171. The method of any one of claims 110-168, wherein the cell is a eukaryotic cell.

172. The method of claim 171, wherein the eukaryotic cell is a yeast cell.

173. The method of claim 171, wherein the eukaryotic cell is a fungal cell.

174. The method of claim 171, wherein the eukaryotic cell is a mammalian cell.

175. The method of claim 171, wherein the eukaryotic cell is an insect cell.

176. The method of claim 171, wherein the eukaryotic cell is a plant cell.

177. The method of any one of claims 110-171, wherein the O-tRNA recognizes a selector codon.

178. The method of claim 177, wherein the selector codon is an amber codon.

179. The method of any one of claims 110-178, wherein the polypeptide comprises an antibody or antigen binding fragment thereof.

180. The method of any one of claims 110-178, wherein the polypeptide comprises human growth hormone.

181. A method of producing a polypeptide comprising at least one nsAA, comprising providing:
i) an O-tRNA comprising a nucleic acid sequence at least 85% identical to the sequence set forth in SEQ ID NO: 1 and comprising a deletion of the cytosine located at nucleic acid position 16 of the O-tRNA; wherein the nucleic acid positions correspond to the sequence set forth in SEQ ID NO: 1; and wherein the O-tRNA is capable of being aminoacylated with at least one non-standard amino acid (nsAA) by an O-RS;
ii) an O-RS; wherein the O-RS aminoacylates the O-tRNA with the nsAA; and
iii) a polynucleotide encoding the polypeptide, wherein the polynucleotide comprises at least one selector codon; and wherein the O-tRNA recognizes the selector codon.

182. The method of claim 181, wherein the nucleic acid sequence is at least 90% identical, at least 91% identical, at least 92% identical, at least 93% identical, at least 94% identical, at least 95% identical, at least 96% identical, at least 97% identical, at least 98% identical, or at least 98.7% identical to the sequence set forth in SEQ ID NO: 1.

183. The method of claim 181 or 182, wherein the O-tRNA comprises an adenine at nucleic acid position 53 and a uracil at nucleic acid position 63, wherein the nucleic acid positions correspond to the sequence set forth in SEQ ID NO:1.

184. The method of claim 183, wherein the O-tRNA comprises a nucleic acid sequence consisting of a sequence set forth in SEQ ID NO: 2.

185. The method of claim 181 or 182, wherein the O-tRNA comprises a cytosine at nucleic acid positions 3 and 6; a uracil at nucleic acid position 7, an adenosine at nucleic acid position 67, and a guanine at nucleic acid positions 68 and 71, wherein the nucleic acid positions correspond to the sequence set forth in SEQ ID NO: 1.

186. The method of claim 185, wherein the O-tRNA comprises a nucleic acid sequence consisting of a sequence set forth in SEQ ID NO: 3.

187. The method of claim 181, wherein the O-tRNA comprises a nucleic acid sequence consisting of a sequence set forth in SEQ ID NO: 4.

188. The method of claim 181 or 182, wherein the O-tRNA comprises the sequence CAG-AGGGCAG at nucleic acid positions 13 to 23, wherein the nucleic acid positions correspond to the sequence set forth in SEQ ID NO: 1.

189. A method of producing a polypeptide comprising at least one non-standard amino acid (nsAA), the method comprising providing
i) an O-tRNA comprising a nucleic acid sequence consisting of a sequence set forth in SEQ ID NO: 36, wherein the sequence does not comprise SEQ ID NO: 1, SEQ ID NO: 37, or SEQ ID NO: 38;
ii) an O-RS; wherein the O-RS aminoacylates the O-tRNA with the nsAA; and
iii) a polynucleotide encoding the polypeptide, wherein the polynucleotide comprises at least one selector codon; and wherein the O-tRNA recognizes the selector codon.

190. The method of claim 186, wherein the O-tRNA comprises a cytosine at position 3.

191. The method of claim 186 or 187, wherein the O-tRNA comprises an adenine at position 4.

192. The method of any one of claims 186-188, wherein the O-tRNA comprises a uracil at position 5.

193. The method of any one of claims 186-189, wherein the O-tRNA comprises a cytosine at position 6.

194. The method of any one of claims 186-190, wherein the O-tRNA comprises a uracil at position 7.

195. The method of any one of claims 186-191, wherein the O-tRNA comprises a guanine at position 46.

196. The method of any one of claims 186-192, wherein the O-tRNA comprises a uracil at position 48.

197. The method of any one of claims 186-193, wherein the O-tRNA comprises an adenine at position 50.

198. The method of any one of claims 186-194, wherein the O-tRNA comprises a guanine at position 51.

199. The method of any one of claims 186-195, wherein the O-tRNA comprises an adenine at position 53.

200. The method of any one of claims 186-196, wherein the O-tRNA comprises a uracil at position 63.

201. The O-tRNA of any one of claims 186-197, wherein the O-tRNA comprises a cytosine at position 65.

202. The method of any one of claims 186-198, wherein the O-tRNA comprises a uracil at position 66.

203. The method of any one of claims 186-199, wherein the O-tRNA comprises an adenine at position 67.

204. The method of any one of claims 186-200, wherein the O-tRNA comprises a guanine at position 68.

205. The method of any one of claims 186-201, wherein the O-tRNA comprises an adenine or a uracil at position 69.

206. The method of any one of claims 186-202, wherein the O-tRNA comprises a uracil at position 70.

207. The method of any one of claims 186-203, wherein the O-tRNA comprises a guanine at position 71.

208. The method of any one of claims 186-204, wherein the O-tRNA comprises a cytosine at position 3, a cytosine at position 6, and a uracil at position 7.

209. The method of any one of claims 186-205, wherein the O-tRNA comprises a guanine at position 46 and a uracil at position 48.

210. The method of any one of claims 186-206, wherein the O-tRNA comprises an adenine at position 67, a guanine at position 68, and a guanine at position 71.

211. The method of any one of claims 186-207, wherein the O-tRNA comprises a nucleic acid sequence consisting of the set forth in any one of SEQ ID NOs: 2-16.

212. The method of any one of claims 186-208, wherein the O-RS comprises an O-RS of an M. jannaschii tyrosyl-tRNA synthetase.

213. The method of claim 212, wherein the O-RS comprises an amino acid sequence consisting of the sequence set forth in SEQ ID NO: 35 or 39.

214. The method of any one of claims 186-209, wherein the nsAA has the structure according to Formula I; and wherein the R group is any substituent other than a corresponding substituent used in the twenty natural amino acids.

215. The method of claim 214, wherein the nsAA has the structure according to Formula I; and wherein the R group comprises an alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amine, or combinations thereof.

216. The method of any one of claims 186-215, wherein the nsAA is selected from the group consisting of an amino acid comprising a photoactivatable cross-linker, a spin-labeled amino acid, a fluorescent amino acid, a metal binding amino acid, a metal containing amino acid, a radioactive amino acid, an amino acid with at least one novel functional group, an amino acid that covalently or noncovalently interacts with other molecules, a photocaged amino acid, a photoisomerizable amino acid, an amino acid comprising biotin or a biotin analogue, a carbohydrate-modified amino acid, an amino acid comprising polyethylene glycol or polyether, a heavy atom substituted amino acid, a chemically cleavable amino acid, a photocleavable amino acid, and combinations thereof.

217. The method of any one of claims 186-216, wherein the nsAA comprises a tyrosine analog.

218. The method of claim 217, wherein the tyrosine analog is selected from the group consisting of a para-substituted tyrosine, an ortho-substituted tyrosine, and a meta-substituted tyrosine.

219. The method of claim 218, wherein the substituted tyrosine comprises a keto group, an acetyl group, a benzoyl group, an amino group, a hydrazine, a hydroxyamine, a thiol group, a carboxy group, an isopropyl group, a methyl group, a branched hydrocarbon, a saturated or unsaturated hydrocarbon, an O-methyl group, a polyether group, a nitro group, or combinations thereof.

220. The method of any one of claims 186-216, wherein the nsAA comprises a glutamine analog.

221. The method of claim 220, wherein the glutamine analog comprises a α-hydroxy derivative, a γ-substituted derivative, a cyclic derivative, an amide substituted glutamine derivative.

222. The method of any one of claims 186-216, wherein the nsAA comprises a phenylalanine analog.

223. The method of claim 222, wherein the phenylalanine analog is an amino-, an isopropyl-, or an O-allyl-containing phenylalanine analog.

224. The method of claim 222 or 223, wherein the phenylalanine analog is selected from the group consisting of a para-substituted phenylalanine, an ortho-substituted phenylalanine, and a meta-substituted phenylalanine.

225. The method of any one of claim 224, wherein the substituent comprises a hydroxy group, a methoxy group, a methyl group, an allyl group, an aldehyde, an azido, an iodo, a bromo, a keto group or an acetyl group.

226. The method of claim 225, wherein the nsAA comprises a para-acetyl phenylalanine.

227. The method of any one of claims 186-216, wherein the nsAA is selected from the group consisting of a p-propargyl phenylalanine, an O-methyl-L-tyrosine, an L-3-(2-naphthyl)alanine, a 3-methylphenylalanine, an O-4-allyl-L-tyrosine, a 4-propyl-L-tyrosine, a tri-O-acetyl-GlcNAcB-serine, an L-Dopa, a fluorinated phenylalanine, an isopropyl-L-phenylalanine, a p-acyl-L-phenylalanine, a p-benzoyl-L-phenylalanine, an L-phosphoserine, a phosphono serine, a phosphonotyrosine, a p-iodo-phenylalanine, a p-bromophenylalanine, a p-amino-L-phenylalanine, and an isopropyl-L-phenylalanine.

228. The method of any one of claims 186-216, wherein the nsAA is selected from the group consisting of 4-acetyl-phenylalanine (AcF), 4-azido-phenylalanine (AzF); 4-propargyloxyphenylalanine (PaF); and 4-aminophenylalanine (AmF).

229. The method of claim 228, wherein the nsAA comprises an 4-acetyl-phenylalanine (AcF).

230. The method of claim 228, wherein the nsAA comprises an 4-azido-phenylalanine (AzF).

231. The method of claim 228, wherein the nsAA comprises an 4-propargyloxyphenylalanine (PaF).

232. The method of claim 228, wherein the nsAA comprises an 4-aminophenylalanine (AmF).

233. The method of claim 186-232, wherein the selector codon is an amber codon.

234. The method of any one of claims 186-233, wherein the polypeptide comprises an antibody or antigen binding fragment thereof.

235. The method of any one of claims 186-234, wherein the polypeptide comprises human growth hormone.

236. The method of any one of claims 186-235, wherein the polypeptide is produced by a cell-free translation system.

237. The method of claim 236, wherein the cell-free translation system is a cell lysate.

238. The method of claim 236, wherein the cell-free translation system is a reconstituted system.
